(19) United States
(12) Patent Application Publication
(10) Pub. No.: US 2006/0210541 A1
(43) Pub. Date: Sep. 21, 2006

(54) Methods for treating and preventing vascular disease

(76) Inventors: Uichi Ikeda, Matsumoto (JP); Tohru Yoshioka, Matsumoto (JP); Yoshikazu Maeda, Kaminokawa-machi (JP); Masahisa Shimpo, Minamikawachi-machi (JP); Kazuyuki Shimada, Utsunomiya (JP); Keiya Ozawa, Minamikawachi-machi (JP); Tatsuya Nomoto, Higashine-shi (JP); Takashi Okada, Minamikawachi-machi (JP); Hiroaki Mizukami, Minamikawachi-machi (JP)

Correspondence Address: ROBINS & PASTERNAK, 1731 EMBARCADERO ROAD, SUITE 230, PALO ALTO, CA, 94303, US

(21) Appl. No.: 11/441,577

(22) Filed: May 26, 2006

Related U.S. Application Data
(62) Division of application No. 10/690,063, filed on Oct. 20, 2003.
(60) Provisional application No. 60/420,348, filed on Oct. 21, 2002.

(51) Int. Cl. A61K 48/00; C12N 15/861

(52) U.S. Cl. 424/93.2; 514/44

(57) ABSTRACT

Methods for treating and/or preventing vascular disease are disclosed. The methods use gene delivery techniques to deliver nucleic acid molecules encoding anti-inflammatory cytokines to a subject.

### CROSS-REFERENCE TO RELATED APPLICATIONS

This application is a divisional application of U.S. patent application Ser. No. 10/690,063, filed Oct. 20, 2003 from which priority is claimed pursuant to 35 U.S.C. §120, which claims the benefit under 35 U.S.C. §19(e) of provisional application 60/420,348, filed Oct. 21, 2002, which applications are hereby incorporated by reference in their entireties.

### TECHNICAL FIELD

The present invention relates generally to gene delivery methods. In particular, the present invention pertains to methods of treating or preventing vascular disease by delivery of nucleic acid encoding anti-inflammatory.

### BACKGROUND

Gene therapy methods are currently being developed that safely and persistently deliver therapeutically effective quantities of gene products to patients. Using these methods, a nucleic acid molecule can be introduced directly into a patient (in vivo gene therapy), or into cells isolated from a patient or a donor, which are then subsequently returned to the patient (ex vivo gene therapy). The introduced nucleic acid then directs the patient's own cells or grafted cells to produce the desired therapeutic product. Gene therapy also allows clinicians to select specific organs or cellular targets (e.g., muscle, blood cells, brain cells, etc.) for therapy.

Nucleic acids may be introduced into a patient's cells in several ways, including viral-mediated gene delivery, naked DNA delivery, and transfection methods. Viral-mediated gene delivery has been used in a majority of gene therapy trials. C. P. Hodgson Biotechnology (1995) 13:222-225. The recombinant viruses most commonly used are based on retrovirus, adenovirus, herpesvirus, pox virus, and adeno-associated virus (AAV). Alternatively, transfection methods may be used for gene delivery. Such methods include chemical transfection techniques, such as calcium phosphate precipitation and liposome-mediated transfection, as well as physical transfection methods such as electroporation. Gene therapy has shown promise for treating a number of diseases using these techniques.

Vascular disease is a major cause of morbidity and mortality in the adult population. For example, arteriolosclerosis, such as atherosclerosis, is responsible for the majority of cases of myocardial and cerebral infarction and represents the principal cause of death in the United States and western Europe. It is now recognized that atherosclerosis causes chronic vascular inflammation such as by endothelial dysfunction, adherence and entry of leukocytes, migration and proliferation of smooth muscle cells, and formation of form cells. These responses alter the normal flow of blood and ultimately lead to accute coronary syndrome and/or stroke.

Interleukin-10 (IL-10) is a pleiotropic cytokine with anti-inflammatory and immunoregulatory functions that plays a critical role in containment and termination of inflammatory responses. For example, IL-10 inhibits the production of proinflammatory cytokines and chemokines such as IL-1, IL-6, MCP-1 and TNF-α, as well as the expression of endothelial adhesion molecules such as ICAM-1, VCAM-1, P-selectin and E-selectin. Experimenters have reported that IL-10 gene therapy reduces pneumonia-induced lung injury (Morrison et al., Infect. Immun. (2000) 68:4752-4758), decreases the severity of rheumatoid arthritis (Ghivizzani et al., Clin. Orthop. (2000) 379 Suppl.:S288-299), decreases inflammatory lung fibrosis (Boehler et al., Hum. Gene Ther. (1998) 9:541-551), inhibits cardiac allograft rejection (Brauner et al., J. Thoracic Cardiovasc. Surg. (1997) 114:923-933), suppresses endotoxemia (Xing et al., Gene Ther. (1997) 4:140-149), prevents and treats colitis (Lindsay et al., J. Immunol. (2001) 166:7625-7633), and reduces contact hypersensitivity (Meng et al., J. Clin. Invest. (1998) 101:1462-1467).

However, the ability of IL-10 gene therapy to treat or prevent vascular disease has not been documented prior to the present invention.

### SUMMARY OF THE INVENTION

The present invention is based on the surprising discovery that vascular disease can be successfully treated and prevented by delivering anti-inflammatory cytokines, such as IL-10, using gene therapy techniques. In particular, the inventors herein have shown in acceptable animal models that gene delivery of anti-inflammatory cytokines, such as IL-10, inhibits the inflammatory response, prevents formation of atherosclerotic lesions, decreases the incidence of stroke, lowers blood pressure in hypertensive subjects, and reduces hypertension-related organ damage.

Accordingly, in one embodiment, the invention is directed to a method of treating or preventing vascular disease in a vertebrate subject comprising administering to said subject a composition comprising a recombinant vector, wherein said recombinant vector comprises a polynucleotide encoding an anti-inflammatory cytokine, operably linked to expression control elements, under conditions that result in expression of the polynucleotide in vivo to provide a therapeutic effect.

In certain embodiments, the anti-inflammatory cytokine is one or more cytokines selected from the group consisting of interleukin-10 (IL-10), interleukin-1 receptor antagonist (IL-1ra), interleukin-4 (IL-4), interleukin-13 (IL-13), tumor necrosis factor soluble receptor (TNFsr), alpha-MSH, and transforming growth factor-beta 1 (TGF-β1).

In additional embodiments of the method, the subject is a human and the anti-inflammatory cytokine is human IL-10.

In yet further embodiments; the recombinant vector is plasmid DNA or a recombinant virus, such as a recombinant adeno-associated virus virion.

In additional embodiments, the administering is by intramuscular injection.

In another embodiment, the invention is directed to a method of treating or preventing vascular disease in a mammalian subject, comprising intramuscularly administering to the subject a composition comprising a recombinant virus, wherein said recombinant virus comprises a polynucleotide encoding IL-10, operably linked to expression control elements, under conditions that result in expression of the polynucleotide in vivo to produce a therapeutic effect.

In certain embodiments, the vascular disease is arteriolosclerosis, atherosclerosis, stroke, and/or hypertension.

In additional embodiments, the mammalian subject is a human and the IL-10 is human IL-10.

In yet further embodiments, the recombinant vector is a recombinant virus, such as a recombinant adeno-associated virion.

In another embodiment, the invention is directed to a method of treating or preventing atherosclerosis in a mammalian subject, comprising intramuscularly administering to the subject a composition comprising a recombinant adeno-associated virus virion, wherein the virion comprises a polynucleotide encoding IL-10, operably linked to expression control elements, under conditions that result in expression of the polynucleotide in vivo to produce a therapeutic effect.

In an additional embodiment, the invention is directed to a method of reducing the incidence of stroke in a mammalian subject, comprising intramuscularly administering to the subject a composition comprising a recombinant adeno-associated virus virion, wherein the virion comprises a polynucleotide encoding IL-10, operably linked to expression control elements, under conditions that result in expression of the polynucleotide in vivo to produce a therapeutic effect.

In still a further embodiment, the invention is directed to a method of treating or preventing hypertension in a mammalian subject, comprising intramuscularly administering to the subject a composition comprising a recombinant adeno-associated virus virion, wherein the virion comprises a polynucleotide encoding IL-10, operably linked to expression control elements, under conditions that result in expression of the polynucleotide in vivo to produce a therapeutic effect.

These and other embodiments of the subject invention will readily occur to those of skill in the art in view of the disclosure herein.

### BRIEF DESCRIPTION OF THE FIGURES

FIGS. 1A and 1B confirm the expression of IL-10 by C2C12 cells transduced with rAAV-IL-10. In FIG. 1A, overexpression of IL-10 was confirmed by ELISA 48 hours after infection of C2C12 cells by rAAV2-IL-10 at the indicated MOIs. FIG. 1B shows a Western blot using anti-IL-10 antibody performed after immunoprecipitation of conditioned medium (CM) and cell lysate (CL).

FIGS. 2A-2C show the effects of rAAV-IL-10 delivered to C2C12 cells on the proinflammatory cytokines IL-6, TNF-α and MCP-1. The results show the average of four different experiments. The mean and SD for each group are presented as histograms. The open bar indicates the LacZ group (control) and the solid bar the IL-10 group.

FIG. 3 shows the detection of secreted IL-10 in serum after injection of varying amounts of rAAV-mIL-10 into the anterior tibial muscles of apoE-deficient mice.

FIG. 4 shows the effects of varying amounts of rAAV-IL-10 delivered to mice on the proinflammatory cytokine MCP-1.

FIGS. 5A-5C show the inhibitory effect of IL-10 on atherosclerosis. Aortic tissue sections were obtained from mice injected with rAAV5-IL-10 virions (1×10¹² particles/body). FIG. 5A shows sections from proximal aorta. FIG. 5B shows lipid lesion formation analysis. The average value for five locations from each animal was used for analysis. FIG. 5C presents the mean and SE for each group as histograms (p<0.01), n=5, LacZ group; n=9, IL-10 group.

FIG. 6 shows MCP-1 levels in mice administered rAAV5-LacZ versus mice given rAAV5-IL-10. Mean and SE for each group are presented as histograms (p,0.05, n=6, LacZ group; n=13, IL-10 group.

FIG. 7 shows a correlation between serum MCP-1 levels and atherosclerotic lesion.

FIGS. 8A and 8B show aortic atherosclerotic lesions stained with antibody against MCP-1. FIG. 8A shows tissue from mice administered rAAV5-LacZ and FIG. 8B shows tissue from mice administered rAAV5-IL-10.

FIG. 9 shows a dose response curve of serum cholesterol level (TC) versus serum IL-10 concentration.

FIG. 10 shows the correlation between serum cholesterol level (TC) and atherosclerotic lesion.

FIG. 11 is a schematic representation of plasmid pWCAGRIL10.

FIG. 12 shows the effect of rAAV-IL-10 on the production of interferon-γ. The results represent the means of two different experiments.

FIGS. 13A and 13B show the serum concentration of IL-10 in SHR-SP rats administered varying amounts of rAAV5-IL-10 (FIG. 13A) or rAAV1-IL-10 (FIG. 13B), as well as results from rats given a control vector or saline. Data are shown as mean±SD.

FIG. 14 shows the systolic blood pressure measurements in SHR-SP rats administered rAAV1-IL-10 or controls. Data are shown as mean±SD.

FIG. 15 shows the correlation between serum IL-10 concentration and blood pressure in SHR-SP rats administered rAAV1-IL-10 or controls.

FIG. 16 shows proteinuria measurements from SHR-SP rats administered rAAV1-IL-10 or controls (n=10 for each group). Data are shown as mean±SD.

FIG. 17 shows the correlation between ejection fraction and serum IL-10 concentration in SHR-SP rats injected with rAAV1-IL-10 or controls.

FIG. 18 shows the percentage of stroke-free animals administered rAAV1-IL-10 or controls (n=10 for each group).

FIG. 19 shows the correlation between serum TGF-β levels and serum IL-10 levels in SHR-SP rats administered rAAV1-IL-10 or controls (n=10 for each group).

### DETAILED DESCRIPTION OF THE INVENTION

The practice of the present invention will employ, unless otherwise indicated, conventional methods of chemistry, biochemistry, recombinant DNA techniques and immunology, within the skill of the art. Such techniques are explained fully in the literature. See, e.g., Fundamental Virology, 2nd Edition, vol. I & II (B. N. Fields and D. M. Knipe, eds.); Handbook of Experimental Immunology, Vols. I-IV (D. M. Weir and C. C. Blackwell eds., Blackwell Scientific Publications); T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.).

All publications, patents and patent applications cited herein, whether supra or infra, are hereby incorporated by reference in their entirety.

### 1. DEFINITIONS

In describing the present invention, the following terms will be employed, and are intended to be defined as indicated below.

It must be noted that, as used in this specification and the appended claims, the singular forms “a”, “an” and “the” include plural referents unless the content clearly dictates otherwise. Thus, for example, reference to “an anti-inflammatory cytokine” includes a mixture of two or more such cytokines, and the like.

By “vascular disease” is meant any disorder of the vasculature, particularly of the blood vessels. Such disorders include, without limitation, hemorrhagic vascular diseases such as hemorrhagic stroke, ischemic vascular diseases, including without limitation, arteriolosclerois, such as atherosclerosis which can lead to ischemic stroke and myocardial infarction, cerebral and cardiac embolism and cerebral thrombosis, hypertension, i.e., elevated arterial blood pressure, such as, but not limited to, essential, primary or idiopathic hypertension, secondary hypertension, malignant hypertension, accelerated hypertension, complicated hypertension, borderline hypertension, etc.

The term “anti-inflammatory cytokine” as used herein refers to a protein that decreases the action or production of one or more proinflammatory cytokines, chemokines or proteins produced by vascular cells, endothelial cells, fibroblasts, muscle, immune cells or other cell types. Such proinflammatory molecules include, without limitation, interleukin-1 beta (IL-1β), tumor necrosis factor-alpha (TNF-α), interleukin-6 (IL-6), inducible nitric oxide synthetase (iNOS), monocyte chemoattractant protein-(MCP-1), and the like. Non-limiting examples of anti-inflammatory cytokines include interleukin-10 (IL-10) including viral IL-10, interleukin-1 receptor antagonist (IL-1ra), interleukin-4 (IL-4), interleukin-13 (IL-13), tumor necrosis factor soluble receptor (TNFsr), alpha-MSH and transforming growth factor-beta 1 (TGF-β1). All of these anti-inflammatory cytokines, as well as fragments, and analogs thereof, which retain the ability to decrease or inhibit the production of proinflammatory cytokines and chemokines such as IL-1, IL-6, MCP-1 and TNF-α, as measured using any of various known assays, including assays described herein, and/or which produce a therapeutic effect in vivo to treat a vascular disease, such as reducing blood pressure, and/or reducing an atherosclerotic area, are intended for use with the present invention.

Thus, the full-length proteins and fragments thereof, as well as proteins with modifications, such as deletions, additions and substitutions (either conservative or non-conservative in nature), to the native sequence, are intended for use herein, so long as the protein maintains the desired activity. These modifications may be deliberate, as through site-directed mutagenesis, or may be accidental, such as through mutations of hosts which produce the proteins or errors due to PCR amplification. Accordingly, active proteins substantially homologous to the parent sequence, e.g., proteins with 70 . . . 80 . . . 85 . . . 90 . . . 95 . . . 98 . . . 99% etc. identity that retain the biological activity, are contemplated for use herein.

The term “analog” refers to biologically active derivatives of the reference molecule, or fragments of such derivatives, that retain activity, as described above. In general, the term “analog” refers to compounds having a native polypeptide sequence and structure with one or more amino acid additions, substitutions and/or deletions, relative to the native molecule. Particularly preferred analogs include substitutions that are conservative in nature, i.e., those substitutions that take place within a family of amino acids that are related in their side chains. Specifically, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, tyrosine. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity. For example, the polypeptide of interest may include up to about 5-10 conservative or non-conservative amino acid substitutions, or even up to about 15-25 or 50 conservative or non-conservative amino acid substitutions, or any number between 5-50, so long as the desired function of the molecule remains intact.

“Homology” refers to the percent identity between two polynucleotide or two polypeptide moieties. Two DNA, or two polypeptide sequences are “substantially homologous” to each other when the sequences exhibit at least about 50%, preferably at least about 75%, more preferably at least about 80%-85%, preferably at least about 90%, and most preferably at least about 95%-98% sequence identity over a defined length of the molecules. As used herein, substantially homologous also refers to sequences showing complete identity to the specified DNA or polypeptide sequence.

In general, “identity” refers to an exact nucleotide-to-nucleotide or amino acid-to-amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353-358, National Biomedical Research Foundation, Washington, DC, which adapts the local homology algorithm of Smith and Waterman Advances in Appl. Math. 2:482-489, 1981 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs are well known in the art.

Alternatively, homology can be determined by hybridization of polynucleotides under conditions which form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

By the term “degenerate variant” is intended a polynucleotide containing changes in the nucleic acid sequence thereof, that encodes a polypeptide having the same amino acid sequence as the polypeptide encoded by the polynucleotide from which the degenerate variant is derived.

A “coding sequence” or a sequence which “encodes” a selected polypeptide, is a nucleic acid molecule which is transcribed (in the case of DNA) and translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. A transcription termination sequence may be located 3′ to the coding sequence.

By “vector” is meant any genetic element, such as a plasmid, phage, transposon, cosmid, chromosome, virus, virion, etc., which is capable of replication when associated with the proper control elements and which can transfer gene sequences to cells. Thus, the term includes cloning and expression vehicles, as well as viral vectors.

By “recombinant vector” is meant a vector that includes a heterologous nucleic acid sequence which is capable of expression in vivo.

By “recombinant virus” is meant a virus that has been genetically altered, e.g., by the addition or insertion of a heterologous nucleic acid construct into the particle.

The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52 :456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous DNA moieties into suitable host cells.

The term “heterologous” as it relates to nucleic acid sequences such as coding sequences and control sequences, denotes sequences that are not normally joined together, and/or are not normally associated with a particular cell. Thus, a “heterologous” region of a nucleic acid construct or a vector is a segment of nucleic acid within or attached to another nucleic acid molecule that is not found in association with the other molecule in nature. For example, a heterologous region of a nucleic acid construct could include a coding sequence flanked by sequences not found in association with the coding sequence in nature. Another example of a heterologous coding sequence is a construct where the coding sequence itself is not found in nature (e.g., synthetic sequences having codons different from the native gene). Similarly, a cell transformed with a construct which is not normally present in the cell would be considered heterologous for purposes of this invention. Allelic variation or naturally occurring mutational events do not give rise to heterologous DNA, as used herein.

A “nucleic acid” sequence refers to a DNA or RNA sequence. The term captures sequences that include any of the known base analogues of DNA and RNA such as, but not limited to 4-acetylcytosine, 8-hydroxy-N6-methyladenosine, aziridinylcytosine, pseudoisocytosine, 5-(carboxyhydroxyl-methyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, -uracil-5-oxyacetic acid methylester, uracil-5-oxyacetic acid, pseudouracil, queosine, 2-thiocytosine, and 2,6-diaminopurine.

The term DNA “control sequences” refers collectively to promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need always be present so long as the selected coding sequence is capable of being replicated, transcribed and translated in an appropriate host cell.

The term “promoter” is used herein in its ordinary sense to refer to a nucleotide region comprising a DNA regulatory sequence, wherein the regulatory sequence is derived from a gene which is capable of binding RNA polymerase and initiating transcription of a downstream (3′-direction) coding sequence. Transcription promoters can include “inducible promoters” (where expression of a polynucleotide sequence operably linked to the promoter is induced by an analyte, cofactor, regulatory protein, etc.), “repressible promoters” (where expression of a polynucleotide sequence operably linked to the promoter is induced by an analyte, cofactor, regulatory protein, etc.), and “constitutive promoters”.

“Operably linked” refers to an arrangement of elements wherein the components so described are configured so as to perform their usual function. Thus, control sequences operably linked to a coding sequence are capable of effecting the expression of the coding sequence. The control sequences need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

By “isolated” when referring to a nucleotide sequence, is meant that the indicated molecule is present in the substantial absence of other biological macromolecules of the same type. Thus, an “isolated nucleic acid molecule which encodes a particular polypeptide” refers to a nucleic acid molecule which is substantially free of other nucleic acid molecules that do not encode the subject polypeptide; however, the molecule may include some additional bases or moieties which do not deleteriously affect the basic characteristics of the composition.

For the purpose of describing the relative position of nucleotide sequences in a particular nucleic acid molecule throughout the instant application, such as when a particular nucleotide sequence is described as being situated “upstream,” “downstream,” “3 prime (3′)” or “5 prime (5′)” relative to another sequence, it is to be understood that it is the position of the sequences in the “sense” or “coding” strand of a DNA molecule that is being referred to as is conventional in the art.

The terms “subject”, “individual” or “patient” are used interchangeably herein and refer to a vertebrate, preferably a mammal. Mammals include, but are not limited to, murines, rodents, simians, humans, farm animals, sport animals and pets.

The terms “effective amount” or “therapeutically effective amount” of a composition or agent, as provided herein, refer to a nontoxic but sufficient amount of the composition or agent to provide the desired “therapeutic effect,” such as to prevent, reduce or reverse symptoms associated with the vascular disorder in question. By “therapeutic effect” is meant a level of expression of one or more heterologous nucleic acid sequences sufficient to alter a component of a disease (or disorder) toward a desired outcome or endpoint, such that a patient's disease or disorder shows improvement, often reflected by the amelioration of a sign or symptom relating to the disease or disorder. The exact amount required will vary from subject to subject, depending on the species, age, and general condition of the subject, the severity of the condition being treated, and the particular macromolecule of interest, mode of administration, and the like. An appropriate “effective” amount in any individual case may be determined by one of ordinary skill in the art using routine experimentation. “Treatment” or “treating” a vascular condition includes: (1) preventing the vascular disease, such as but not limited to, preventing atherosclerosis, stroke and/or high blood pressure or (2) causing vascular disorders to develop or to occur at lower rates in a subject that may be exposed to agents or conditions causing such disorders or that is predisposed to such disorders, (3) reducing the vascular condition in question, such as reducing an atherosclerotic area or reducing blood pressure.

### 2. MODES OF CARRYING OUT THE INVENTION

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

Central to the present invention is the discovery that gene therapy using genes encoding anti-inflammatory cytokines serves to treat or prevent vascular disorders in vertebrate subjects. Advantages of this approach to the control of such disorders are numerous. For example, sustained delivery of an anti-inflammatory agent can be achieved with only a single administration of a composition according to the invention. Thus, patient compliance is greatly enhanced. Gene therapy techniques can be used alone or in conjunction with traditional drug and protein delivery techniques. Thus, compounds traditionally used to treat vascular diseases can also be administered to the subject.

In order to further an understanding of the invention, a more detailed discussion is provided below regarding anti-inflammatory cytokines, as well as various gene delivery methods for use with the present invention.

Anti-Inflammatory Cytokines

As explained above, the present invention makes use of anti-inflammatory cytokines to treat or prevent vascular disease. Particularly preferred anti-inflammatory cytokines include interleukin-10 (IL-10), interleukin-1 receptor antagonist (IL-1ra), interleukin-4 (IL-4), interleukin-13 (IL-13), tumor necrosis factor soluble receptor (TNFsr), alpha-MSH and transforming growth factor-beta 1 (TGF-β1). The native molecules, as well as fragments and analogs thereof, which retain biological activity as defined above, are intended for use with the present invention. Moreover, sequences derived from any of numerous species can be used with the present invention, depending on the animal to be treated.

Nucleotide and amino acid sequences of each of these anti-inflammatory cytokines and variants thereof, from several animal species are well known. For example, IL-10 has been isolated from a number of animal and viral species. IL-10 for use herein includes IL-10 from any of these various species. Non-limiting examples of viral IL-10 include the IL-10 homologues isolated from the herpesviruses such as from Epstein-Barr virus (see, e.g., Moore et al., Science (1990) 248:1230-1234; Hsu et al., Science (1990) 250:830-832; Suzuki et al., J. Exp. Med. (1995) 182:477-486), Cytomegalovirus (see, e.g., Lockridge et al., Virol. (2000) 268:272-280; Kotenko et al., Proc. Natl. Acad. Sci. USA (2000) 97:1695-1700), and equine herpesvirus (see, e.g., Rode et al., Virus Genes (1993) 7:111-116), as well as the IL-10 homologue from the OrF virus (see, e.g., Imlach et al., J. Gen. Virol. (2002) 83:1049-1058 and Fleming et al., Virus Genes (2000) 21:85-95). Representative, non-limiting examples of other IL-10 sequences for use with the present invention include the sequences described in NCBI accession numbers NM000572, U63015, AF418271, AF247603, AF247604, AF247606, AF247605, AY029171, UL16720 (all human sequences); NM012854, L02926, X60675 (rat); NM010548, AF307012, M37897, M84340 (all mouse sequences); U38200 (equine); U39569, AF060520 (feline sequences); U00799 (bovine); U11421, Z29362 (ovine sequences); L26031, L26029 (macaque sequences); AF294758 (monkey); U33843 (canine); AF088887, AF068058 (rabbit sequences); AF012909, AF120030 (woodchuck sequences); AF026277 (possum); AF097510 (guinea pig); U11767 (deer); L37781 (gerbil); AB107649 (llama and camel).

Non-limiting examples of IL-1ra sequences for use with the present invention include the sequences described in NCBI accession numbers NM173843, NM173842, NM173841, NM000577, AY196903, BC009745, AJ005835, X64532, M63099, X77090, X52015, M55646 (all human sequences); NM174357, AB005148 (bovine sequences); NM031167, S64082, M57525, M644044 (mouse sequences); D21832, 568977, M57526 (rabbit sequences); SEG AB045625S, M63101 (rat sequences); AF216526, AY026462 (canine sequences); U92482, D83714 (equine sequences); AB038268 (dolphin).

Non-limiting examples of IL-4 sequences for use with the present invention include the sequences described in NCBI accession numbers NM172348, AF395008, AB015021, X16710, A00076, M13982, NM000589 (all human sequences); BC027514, NM021283, AF352783, M25892 (mouse sequences); NM173921, AH003241, M84745, M77120 (bovine sequences); AY130260 (chimp); AF097321, L26027 (monkey); AY096800, AF172168, Z11897, M96845 (ovine sequences); AF035404, AF305617 (equine sequences); AF239917, AF187322, AF054833, AF104245 (canine sequences); X16058 (rat); AF046213 (hamster); L07081 (cervine); U39634, X87408 (feline); X68330, L12991 (porcine sequences); U34273 (goat); AB020732 (dolphin); L37779 (gerbil); AF068058, AF169169 (rabbit sequences); AB107648 (llama and camel).

Non-limiting examples of IL-13 sequences for use with the present invention include the sequences described in NCBI accession numbers NM002188, U10307, AF377331, X69079 (all human sequences); NM053828, L26913 (rat sequences); AF385626, AF385625 (porcine sequences); AF244915 (canine); NM174089 (bovine); AY244790 (monkey); NM008355 (mouse); AB107658 (camel); AB107650 (llama).

Non-limiting examples of TGF-β1 sequences for use with the present invention include the sequences described in NCBI accession numbers NM000660, BD0097505, BD0097504, BD0097503, BD0097502 (all human sequences); NM021578, X52498 (rat sequences); AJ009862, NM011577, BC013738, M57902 (mouse sequences); AF461808, X12373, M23703 (porcine sequences); AF175709, X99438 (equine sequences); X76916 (ovine); X60296 (hamster); L34956 (canine).

Non-limiting examples of alpha-MSH sequences for use with the present invention include the sequences described in NCBI accession number NM 000939 (human); NM17451 (bovine); NM 008895 (mouse); and M 11346 (xenopus).

Non-limiting examples of TNF receptor sequences for use with the present invention include the sequences described in NCBI accession numbers X55313, M60275, M63121, NM152942, NM001242, NM152877, NM152876, NM152875, NM152874, NM152873, NM152872, NM152871, NM000043, NM 001065, NM001066, NM148974, NM148973, NM148972, NM148971, NM148970, NM148969, NM148968, NM148967, NM148966, NM148965, NM003790, NM032945, NM003823, NM001243, NM152854, NM001250 (all human sequences); NM013091, M651122 (rat sequences).

Polynucleotides encoding the desired anti-inflammatory cytokine for use with the present invention can be made using standard techniques of molecular biology. For example, polynucleotide sequences coding for the above-described molecules can be obtained using recombinant methods, such as by screening cDNA and genomic libraries from cells expressing the gene, or by deriving the gene from a vector known to include the same. The gene of interest can also be produced synthetically, rather than cloned, based on the known sequences. The molecules can be designed with appropriate codons for the particular sequence. The complete sequence is then assembled from overlapping oligonucleotides prepared by standard methods and assembled into a complete coding sequence. See, e.g., Edge, Nature (1981) 292:756; Nambair et al., Science (1984) 223:1299; and Jay et al., J. Biol. Chem. (1984) 259:6311.

Thus, particular nucleotide sequences can be obtained from vectors harboring the desired sequences or synthesized completely or in part using various oligonucleotide synthesis techniques known in the art, such as site-directed mutagenesis and polymerase chain reaction (PCR) techniques where appropriate. See, e.g., Sambrook, supra. One method of obtaining nucleotide sequences encoding the desired sequences is by annealing complementary sets of overlapping synthetic oligonucleotides produced in a conventional, automated polynucleotide synthesizer, followed by ligation with an appropriate DNA ligase and amplification of the ligated nucleotide sequence via PCR. See, e.g., Jayaraman et al., Proc. Natl. Acad. Sci. USA (1991) 88:4084-4088. Additionally, oligonucleotide-directed synthesis (Jones et al., Nature (1986) 54:75-82), oligonucleotide directed mutagenesis of preexisting nucleotide regions (Riechmann et al., Nature (1988) 332:323-327 and Verhoeyen et al., Science (1988) 239:1534-1536), and enzymatic filling-in of gapped oligonucleotides using T₄ DNA polymerase (Queen et al., Proc. Natl. Acad. Sci. USA (1989) 86:10029-10033) can be used to provide molecules for use in the subject methods.

Gene Delivery Techniques

Anti-inflammatory genes as described above, are delivered to the subject in question using any of several gene-delivery techniques. Several methods for gene delivery are known in the art. As described further below, genes can be delivered either directly to the mammalian subject or, alternatively, delivered ex vivo, to cells derived from the subject and the cells reimplanted in the subject.

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems have been described. See, e.g., U.S. Pat. No. 5,219,740; Miller and Rosman, BioTechniques (1989) 7:980-990; Miller, A. D., Human Gene Therapy (1990) 1:5-14; Scarpa et al., Virology (1991) 180:849-852; Burns et al., Proc. Natl. Acad. Sci. USA (1993) 90:8033-8037; and Boris-Lawrie and Temin, Cur. Opin. Genet. Develop. (1993) 3:102-109. Replication-defective murine retroviral vectors are widely utilized gene transfer vectors. Murine leukemia retroviruses include a single strand RNA complexed with a nuclear core protein and polymerase (pol) enzymes encased by a protein core (gag) and surrounded by a glycoprotein envelope (env) that determines host range. The genomic structure of retroviruses include gag, pol, and env genes enclosed at the 5′ and 3′ long terminal repeats (LTRs). Retroviral vector systems exploit the fact that a minimal vector containing the 5′ and 3′ LTRs and the packaging signal are sufficient to allow vector packaging and infection and integration into target cells provided that the viral structural proteins are supplied in trans in the packaging cell line. Fundamental advantages of retroviral vectors for gene transfer include efficient infection and gene expression in most cell types, precise single copy vector integration into target cell chromosomal DNA and ease of manipulation of the retroviral genome.

A number of adenovirus vectors have also been described. Unlike retroviruses which integrate into the host genome, adenoviruses persist extrachromosomally thus minimizing the risks associated with insertional mutagenesis (Haj-Ahmad and Graham, J. Virol. (1986) 57:267-274; Bett et al., J. Virol. (1993) 67:5911-5921; Mittereder et al., Human Gene Therapy (1994) 5:717-729; Seth et al., J. Virol. (1994) 68:933-940; Barr et al., Gene Therapy (1994) 1:51-58; Berkner, K. L. BioTechniques (1988) 6:616-629; and Rich et al., Human Gene Therapy (1993) 4:461-476). Adenovirus vectors for use in the subject methods are described in more detail below.

Additionally, various adeno-associated virus (AAV) vector systems have been developed for gene delivery. AAV vectors can be readily constructed using techniques well known in the art. See, e.g., U.S. Pat. Nos. 5,173,414 and 5,139,941; International Publication Nos. WO 92/01070 (published 23 Jan. 1992) and WO 93/03769 (published 4 Mar. 1993); Lebkowski et al., Molec. Cell. Biol. (1988) 8:3988-3996; Vincent et al., Vaccines 90 (1990) (Cold Spring Harbor Laboratory Press); Carter, B. J. Current Opinion in Biotechnology (1992) 3:533-539; Muzyczka, N. Current Topics in Microbiol. and Immunol. (1992) 158:97-129; Kotin, R. M. Human Gene Therapy (1994) 5:793-801; Shelling and Smith, Gene Therapy (1994) 1:165-169; and Zhou et al., J. Exp. Med. (1994) 179:1867-1875. AAV vector systems are also described in further detail below.

Additional viral vectors which will find use for delivering the nucleic acid molecules of interest include those derived from the pox family of viruses, including vaccinia virus and avian poxvirus. By way of example, vaccinia virus recombinants expressing the genes can be constructed as follows. The DNA encoding the particular polypeptide is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the protein into the viral genome. The resulting TK-recombinant can be selected by culturing the cells in the presence of 5-bromodeoxyuridine and picking viral plaques resistant thereto.

Alternatively, avipoxviruses, such as the fowlpox and canarypox viruses, can also be used to deliver the genes. Recombinant avipox viruses, expressing immunogens from mammalian pathogens, are known to confer protective immunity when administered to non-avian species. The use of an avipox vector is particularly desirable in human and other mammalian species since members of the avipox genus can only productively replicate in susceptible avian species and therefore are not infective in mammalian cells. Methods for producing recombinant avipoxviruses are known in the art and employ genetic recombination, as described above with respect to the production of vaccinia viruses. See, e.g., WO 91/12882; WO 89/03429; and WO 92/03545.

Molecular conjugate vectors, such as the adenovirus chimeric vectors described in Michael et al., J. Biol. Chem. (1993) 268:6866-6869 and Wagner et al., Proc. Natl. Acad. Sci. USA (1992) 89:6099-6103, can also be used for gene delivery.

Members of the Alphavirus genus, such as but not limited to vectors derived from the Sindbis and Semliki Forest viruses, will also find use as viral vectors for delivering the anti-inflammatory cytokine gene. For a description of Sinbus-virus derived vectors useful for the practice of the instant methods, see, Dubensky et al., J. Virol. (1996) 70:508-519; and International Publication Nos. WO 95/07995 and WO 96/17072.

Alternatively, the anti-inflammatory cytokines can be delivered without the use of viral vectors, such as by using plasmid-based nucleic acid delivery systems as described in U.S. Pat. Nos. 6,413,942; 6,214,804; 5,580,859; 5,589,466; 5,763,270; and 5,693,622, all incorporated herein by reference in their entireties. Plasmids will include the gene of interest operably linked to control elements that direct the expression of the protein product in vivo. Such control elements are well known in the art.

Adeno-Associated Virus Gene Delivery Systems

In a preferred embodiment of the subject invention, a nucleotide sequence encoding the anti-inflammatory cytokine is inserted into an adeno-associated virus-based expression vector. Adeno-associated virus (AAV) has been used with success to deliver a wide variety of genes for gene therapy. The AAV genome is a linear, single-stranded DNA molecule containing about 4681 nucleotides. The AAV genome generally comprises an internal, nonrepeating genome flanked on each end by inverted terminal repeats (ITRs). The ITRs are approximately 145 base pairs (bp) in length. The ITRs have multiple functions, including providing origins of DNA replication, and packaging signals for the viral genome. The internal nonrepeated portion of the genome includes two large open reading frames, known as the AAV replication (rep) and capsid (cap) genes. The rep and cap genes code for viral proteins that allow the virus to replicate and package into a virion. In particular, a family of at least four viral proteins are expressed from the AAV rep region, Rep 78, Rep 68, Rep 52, and Rep 40, named according to their apparent molecular weight. The AAV cap region encodes at least three proteins, VPI, VP2, and VP3.

AAV has been engineered to deliver genes of interest by deleting the internal nonrepeating portion of the AAV genome (i.e., the rep and cap genes) and inserting a heterologous gene (in this case, the gene encoding the anti-inflammatory cytokine) between the ITRs. The heterologous gene is typically functionally linked to a heterologous promoter (constitutive, cell-specific, or inducible) capable of driving gene expression in the patient's target cells under appropriate conditions. Termination signals, such as polyadenylation sites, can also be included.

AAV is a helper-dependent virus; that is, it requires coinfection with a helper virus (e.g., adenovirus, herpesvirus or vaccinia), in order to form AAV virions. In the absence of coinfection with a helper virus, AAV establishes a latent state in which the viral genome inserts into a host cell chromosome, but infectious virions are not produced. Subsequent infection by a helper virus “rescues” the integrated genome, allowing it to replicate and package its genome into an infectious AAV virion. While AAV can infect cells from different species, the helper virus must be of the same species as the host cell. Thus, for example, human AAV will replicate in canine cells coinfected with a canine adenovirus.

Recombinant AAV virions comprising the anti-inflammatory cytokine coding sequence may be produced using a variety of art-recognized techniques described more fully below. Wild-type AAV and helper viruses may be used to provide the necessary replicative functions for producing rAAV virions (see, e.g., U.S. Pat. No. 5,139,941, incorporated herein by reference in its entirety). Alternatively, a plasmid, containing helper function genes, in combination with infection by one of the well-known helper viruses can be used as the source of replicative functions (see e.g., U.S. Pat. No. 5,622,856 and U.S. Pat. No. 5,139,941, both incorporated herein by reference in their entireties). Similarly, a plasmid, containing accessory function genes can be used in combination with infection by wild-type AAV, to provide the necessary replicative functions. These three approaches, when used in combination with a rAAV vector, are each sufficient to produce rAAV virions. Other approaches, well known in the art, can also be employed by the skilled artisan to produce rAAV virions.

In a preferred embodiment of the present invention, a triple transfection method (described in detail in U.S. Pat. No. 6,001,650, incorporated by reference herein in its entirety) is used to produce rAAV virions because this method does not require the use of an infectious helper virus, enabling rAAV virions to be produced without any detectable helper virus present. This is accomplished by use of three vectors for rAAV virion production: an AAV helper function vector, an accessory function vector, and a rAAV expression vector. One of skill in the art will appreciate, however, that the nucleic acid sequences encoded by these vectors can be provided on two or more vectors in various combinations.

As explained herein, the AAV helper function vector encodes the “AAV helper function” sequences (i.e., rep and cap), which function in trans for productive AAV replication and encapsidation. Preferably, the AAV helper function vector supports efficient AAV vector production without generating any detectable wt AAV virions (i.e., AAV virions containing functional rep and cap genes). An example of such a vector, pHLP19, is described in U.S. Pat. No. 6,001,650, incorporated herein by reference in its entirety. The rep and cap genes of the AAV helper function vector can be derived from any of the known AAV serotypes, as explained above. For example, the AAV helper function vector may have a rep gene derived from AAV-2 and a cap gene derived from AAV-6; one of skill in the art will recognize that other rep and cap gene combinations are possible, the defining feature being the ability to support rAAV virion production.

The accessory function vector encodes nucleotide sequences for non-AAV-derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the well-known helper viruses such as adenovirus, herpesvirus (other than herpes simplex virus type-1), and vaccinia virus. In a preferred embodiment, the accessory function plasmid pLadeno5 is used (details regarding pLadeno5 are described in U.S. Pat. No. 6,004,797, incorporated herein by reference in its entirety). This plasmid provides a complete set of adenovirus accessory functions for AAV vector production, but lacks the components necessary to form replication-competent adenovirus.

In order to further an understanding of AAV, a more detailed discussion is provided below regarding recombinant AAV expression vectors and AAV helper and accessory functions

Recombinant AAV Expression Vectors

Recombinant AAV (rAAV) expression vectors are constructed using known techniques to at least provide as operatively linked components in the direction of transcription, control elements including a transcriptional initiation region, the anti-inflammatory polynucleotide of interest and a transcriptional termination region. The control elements are selected to be functional in a mammalian muscle cell. The resulting construct which contains the operatively linked components is bounded (5′ and 3′) with functional AAV ITR sequences.

The nucleotide sequences of AAV ITR regions are known. See, e.g., Kotin, R. M. (1994) Human Gene Therapy 5:793-801; Berns, K. I. “Parvoviridae and their Replication” in Fundamental Virology, 2nd Edition, (B. N. Fields and D. M. Knipe, eds.) for the AAV-2 sequence. AAV ITRs used in the vectors of the invention need not have a wild-type nucleotide sequence, and may be altered, e.g., by the insertion, deletion or substitution of nucleotides. Additionally, AAV ITRs may be derived from any of several AAV serotypes, including without limitation, AAV-1, AAV-2, AAV-3, AAV-4, AAV-5, AAV-6, AAV-7 and AAV-8, etc. Furthermore, 5′ and 3′ ITRs which flank a selected nucleotide sequence in an AAV expression vector need not necessarily be identical or derived from the same AAV serotype or isolate, so long as they function as intended, i.e., to allow for excision and rescue of the sequence of interest from a host cell genome or vector, and to allow integration of the DNA molecule into the recipient cell genome when AAV Rep gene products are present in the cell.

Suitable polynucleotide molecules for use in AAV vectors will be less than about 5 kilobases (kb) in size. The selected polynucleotide sequence is operably linked to control elements that direct the transcription or expression thereof in the subject in vivo. Such control elements can comprise control sequences normally associated with the selected gene. Alternatively, heterologous control sequences can be employed. Useful heterologous control sequences generally include those derived from sequences encoding mammalian or viral genes. Examples include, but are not limited to, neuron-specific enolase promoter, a GFAP promoter, the SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, the CAG promoter, synthetic promoters, hybrid promoters, and the like. In addition, sequences derived from nonviral genes, such as the murine metallothionein gene, will also find use herein. Such promoter sequences are commercially available from, e.g., Stratagene (San Diego, Calif.).

The AAV expression vector which harbors the polynucleotide molecule of interest bounded by AAV ITRs, can be constructed by directly inserting the selected sequence(s) into an AAV genome which has had the major AAV open reading frames (“ORFs”) excised therefrom. Other portions of the AAV genome can also be deleted, so long as a sufficient portion of the ITRs remain to allow for replication and packaging functions. Such constructs can be designed using techniques well known in the art. See, e.g., U.S. Pat. Nos. 5,173,414 and 5,139,941; International Publication Nos. WO 92/01070 (published 23 Jan. 1992) and WO 93/03769 (published 4 Mar. 1993); Lebkowski et al. (1988) Molec. Cell. Biol. 8:3988-3996; Vincent et al. (1990) Vaccines 90 (Cold Spring Harbor Laboratory Press); Carter (1992) Current Opinion in Biotechnology 3:533-539; Muzyczka (1992) Current Topics in Microbiol. and Immunol. 158:97-129; Kotin (1994) Human Gene Therapy 5:793-801; Shelling and Smith (1994) Gene Therapy 1:165-169; and Zhou et al. (1994) J. Exp. Med. 179:1867-1875.

Alternatively, AAV ITRs can be excised from the viral genome or from an AAV vector containing the same and fused 5′ and 3′ of a selected nucleic acid construct that is present in another vector using standard ligation techniques, such as those described in Sambrook et al., supra. For example, ligations can be accomplished in 20 mM Tris-Cl pH 7.5, 10 mM MgCl2, 10 mM DTT, 33 μg/ml BSA, 10 mM-50 mM NaCl, and either 40 μM ATP, 0.01-0.02 (Weiss) units T4 DNA ligase at 0° C. (for “sticky end” ligation) or 1 mM ATP, 0.3-0.6 (Weiss) units T4 DNA ligase at 14° C. (for “blunt end” ligation). Intermolecular “sticky end” ligations are usually performed at 30-100 μg/ml total DNA concentrations (5-100 nM total end concentration). AAV vectors which contain ITRs have been described in, e.g., U.S. Pat. No. 5,139,941. In particular, several AAV vectors are described therein which are available from the American Type Culture Collection (“ATCC”) under Accession Numbers 53222, 53223, 53224, 53225 and 53226.

For the purposes of the invention, suitable host cells for producing rAAV virions from the AAV expression vectors include microorganisms, yeast cells, insect cells, and mammalian cells, that can be, or have been, used as recipients of a heterologous DNA molecule and that are capable of growth in, for example, suspension culture, a bioreactor, or the like. The term includes the progeny of the original cell which has been transfected. Thus, a “host cell” as used herein generally refers to a cell which has been transfected with an exogenous DNA sequence. Cells from the stable human cell line, 293 (readily available through, e.g., the American Type Culture Collection under Accession Number ATCC CRL1573) are preferred in the practice of the present invention. Particularly, the human cell line 293 is a human embryonic kidney cell line that has been transformed with adenovirus type-5 DNA fragments (Graham et al. (1977) J. Gen. Virol. 36:59), and expresses the adenoviral E1a and E1b genes (Aiello et al. (1979) Virology 94:460). The 293 cell line is readily transfected, and provides a particularly convenient platform in which to produce rAAV virions.

AAV Helper Functions

Host cells containing the above-described AAV expression vectors must be rendered capable of providing AAV helper functions in order to replicate and encapsidate the nucleotide sequences flanked by the AAV ITRs to produce rAAV virions. AAV helper functions are generally AAV-derived coding sequences which can be expressed to provide AAV gene products that, in turn, function in trans for productive AAV replication. AAV helper functions are used herein to complement necessary AAV functions that are missing from the AAV expression vectors. Thus, AAV helper functions include one, or both of the major AAV ORFs, namely the rep and cap coding regions, or functional homologues thereof.

By “AAV rep coding region” is meant the art-recognized region of the AAV genome which encodes the replication proteins Rep 78, Rep 68, Rep 52 and Rep 40. These Rep expression products have been shown to possess many functions, including recognition, binding and nicking of the AAV origin of DNA replication, DNA helicase activity and modulation of transcription from AAV (or other heterologous) promoters. The Rep expression products are collectively required for replicating the AAV genome. For a description of the AAV rep coding region, see, e.g., Muzyczka, N. (1992) Current Topics in Microbiol. and Immunol. 158:97-129; and Kotin, R. M. (1994) Human Gene Therapy 5:793-801. Suitable homologues of the AAV rep coding region include the human herpesvirus 6 (HHV-6) rep gene which is also known to mediate AAV-2 DNA replication (Thomson et al. (1994) Virology 204:304-311).

By “AAV cap coding region” is meant the art-recognized region of the AAV genome which encodes the capsid proteins VP1, VP2, and VP3, or functional homologues thereof. These Cap expression products supply the packaging functions which are collectively required for packaging the viral genome. For a description of the AAV cap coding region, see, e.g., Muzyczka, N. and Kotin, R. M. (supra).

AAV helper functions are introduced into the host cell by transfecting the host cell with an AAV helper construct either prior to, or concurrently with, the transfection of the AAV expression vector. AAV helper constructs are thus used to provide at least transient expression of AAV rep and/or cap genes to complement missing AAV functions that are necessary for productive AAV infection. AAV helper constructs lack AAV ITRs and can neither replicate nor package themselves.

These constructs can be in the form of a plasmid, phage, transposon, cosmid, virus, or virion. A number of AAV helper constructs have been described, such as the commonly used plasmids pAAV/Ad and pIM29+45 which encode both Rep and Cap expression products. See, e.g., Samulski et al. (1989) J. Virol. 63:3822-3828; and McCarty et al. (1991) J. Virol. 65:2936-2945. A number of other vectors have been described which encode Rep and/or Cap expression products. See, e.g., U.S. Pat. No. 5,139,941.

AAV Accessory Functions

The host cell (or packaging cell) must also be rendered capable of providing nonAAV-derived functions, or “accessory functions,” in order to produce rAAV virions. Accessory functions are nonAAV-derived viral and/or cellular functions upon which AAV is dependent for its replication. Thus, accessory functions include at least those nonAAV proteins and RNAs that are required in AAV replication, including those involved in activation of AAV gene transcription, stage specific AAV mRNA splicing, AAV DNA replication, synthesis of Cap expression products and AAV capsid assembly. Viral-based accessory functions can be derived from any of the known helper viruses.

In particular, accessory functions can be introduced into and then expressed in host cells using methods known to those of skill in the art. Typically, accessory functions are provided by infection of the host cells with an unrelated helper virus. A number of suitable helper viruses are known, including adenoviruses; herpesviruses such as herpes simplex virus types 1 and 2; and vaccinia viruses. Nonviral accessory functions will also find use herein, such as those provided by cell synchronization using any of various known agents. See, e.g., Buller et al. (1981) J. Virol. 40:241-247; McPherson et al. (1985) Virology 147:217-222; Schlehofer et al. (1986) Virology 152:110-117.

Alternatively, accessory functions can be provided using an accessory function vector as defined above. See, e.g., U.S. Pat. No. 6,004,797 and International Publication No. WO 01/83797, incorporated herein by reference in its entirety. Nucleic acid sequences providing the accessory functions can be obtained from natural sources, such as from the genome of an adenovirus particle, or constructed using recombinant or synthetic methods known in the art. As explained above, it has been demonstrated that the full-complement of adenovirus genes are not required for accessory helper functions. In particular, adenovirus mutants incapable of DNA replication and late gene synthesis have been shown to be permissive for AAV replication. Ito et al., (1970) J. Gen. Virol. 9:243; Ishibashi et al, (1971) Virology 45:317. Similarly, mutants within the E2B and E3 regions have been shown to support AAV replication, indicating that the E2B and E3 regions are probably not involved in providing accessory functions. Carter et al., (1983) Virology 126:505. However, adenoviruses defective in the E1 region, or having a deleted E4 region, are unable to support AAV replication. Thus, E1A and E4 regions are likely required for AAV replication, either directly or indirectly. Laughlin et al., (1982) J. Virol. 41:868; Janik et al., (1981) Proc. Natl. Acad. Sci. USA 78:1925; Carter et al., (1983) Virology 126:505. Other characterized Ad mutants include: E1B (Laughlin et al. (1982), supra; Janik et al. (1981), supra; Ostrove et al., (1980) Virology 104:502); E2A (Handa et al., (1975) J. Gen. Virol. 29:239; Strauss et al., (1976) J. Virol. 17:140; Myers et al., (1980) J. Virol. 35:665; Jay et al., (1981) Proc. Natl. Acad. Sci. USA 78:2927; Myers et al., (1981) J. Biol. Chem. 256:567); E2B (Carter, Adeno-Associated Virus Helper Functions, in I CRC Handbook of Parvoviruses (P. Tijssen ed., 1990)); E3 (Carter et al. (1983), supra); and E4 (Carter et al.(1983), supra; Carter (1995)). Although studies of the accessory functions provided by adenoviruses having mutations in the E1B coding region have produced conflicting results, Samulski et al., (1988) J. Virol. 62:206-210, recently reported that E1B55k is required for AAV virion production, while E1B19k is not. In addition, International Publication WO 97/17458 and Matshushita et al., (1998) Gene Therapy 5:938-945, describe accessory function vectors encoding various Ad genes. Particularly preferred accessory function vectors comprise an adenovirus VA RNA coding region, an adenovirus E4 ORF6 coding region, an adenovirus E2A 72 kD coding region, an adenovirus E1A coding region, and an adenovirus E1B region lacking an intact E1B55k coding region. Such vectors are described in International Publication No. WO 01/83797.

As a consequence of the infection of the host cell with a helper virus, or transfection of the host cell with an accessory function vector, accessory functions are expressed which transactivate the AAV helper construct to produce AAV Rep and/or Cap proteins. The Rep expression products excise the recombinant DNA (including the DNA of interest) from the AAV expression vector. The Rep proteins also serve to duplicate the AAV genome. The expressed Cap proteins assemble into capsids, and the recombinant AAV genome is packaged into the capsids. Thus, productive AAV replication ensues, and the DNA is packaged into rAAV virions. A “recombinant AAV virion,” or “rAAV virion” is defined herein as an infectious, replication-defective virus including an AAV protein shell, encapsidating a heterologous nucleotide sequence of interest which is flanked on both sides by AAV ITRs.

Following recombinant AAV replication, rAAV virions can be purified from the host cell using a variety of conventional purification methods, such as column chromatography, CsCl gradients, and the like. For example, a plurality of column purification steps can be used, such as purification over an anion exchange column, an affinity column and/or a cation exchange column. See, for example, International Publication No. WO 02/12455. Further, if infection is employed to express the accessory functions, residual helper virus can be inactivated, using known methods. For example, adenovirus can be inactivated by heating to temperatures of approximately 60 C for, e.g., 20 minutes or more. This treatment effectively inactivates only the helper virus since AAV is extremely heat stable while the helper adenovirus is heat labile.

The resulting rAAV virions containing the nucleotide sequence of interest can then be used for gene delivery using the techniques described below.

Adenovirus Gene Delivery Systems

In another preferred embodiment, the gene of interest is delivered using an adenovirus gene delivery system. The adenovirus genome is a linear double-stranded DNA molecule of approximately 36,000 base pairs with the 55-kDa terminal protein covalently bound to the 5′ terminus of each strand. Adenoviral (“Ad”) DNA contains identical Inverted Terminal Repeats (“ITRs”) of about 100 base pairs with the exact length depending on the serotype. The viral origins of replication are located within the ITRs exactly at the genome ends. DNA synthesis occurs in two stages. First, replication proceeds by strand displacement, generating a daughter duplex molecule and a parental displaced strand. The displaced strand is single-stranded and can form a “panhandle” intermediate, which allows replication initiation and generation of a daughter duplex molecule. Alternatively, replication can proceed from both ends of the genome simultaneously, obviating the requirement to form the panhandle structure.

During the productive infection cycle, the viral genes are expressed in two phases: the early phase, which is the period up to viral DNA replication, and the late phase, which coincides with the initiation of viral DNA replication. During the early phase only the early gene products, encoded by regions E1, E2, E3 and E4, are expressed, which carry out a number of functions that prepare the cell for synthesis of viral structural proteins. During the late phase, late viral gene products are expressed in addition to the early gene products and host cell DNA and protein synthesis are shut off. Consequently, the cell becomes dedicated to the production of viral DNA and of viral structural proteins.

The E1 region of adenovirus is the first region expressed after infection of the target cell. This region consists of two transcriptional units, the E1A and E1B genes. The main functions of the E1A gene products are to induce quiescent cells to enter the cell cycle and resume cellular DNA synthesis, and to transcriptionally activate the E1B gene and the other early regions (E2, E3, E4). Transfection of primary cells with the E1A gene alone can induce unlimited proliferation (immortalization), but does not result in complete transformation. However, expression of E1A in most cases results in induction of programmed cell death (apoptosis), and only occasionally immortalization. Coexpression of the E1B gene is required to prevent induction of apoptosis and for complete morphological transformation to occur. In established immortal cell lines, high level expression of E1A can cause complete transformation in the absence of E1B.

The E1B-encoded proteins assist E1A in redirecting the cellular functions to allow viral replication. The E1B 55 kD and E4 33 kD proteins, which form a complex that is essentially localized in the nucleus, function in inhibiting the synthesis of host proteins and in facilitating the expression of viral genes. Their main influence is to establish selective transport of viral mRNAs from the nucleus to the cytoplasm, concomittantly with the onset of the late phase of infection. The E1B 21 kD protein is important for correct temporal control of the productive infection cycle, thereby preventing premature death of the host cell before the virus life cycle has been completed.

Adenoviral-based vectors express gene product peptides at high levels. Adenoviral vectors have high efficiencies of infectivity, even with low titers of virus. Additionally, the virus is fully infective as a cell-free virion so injection of producer cell lines are not necessary. Adenoviral vectors achieve long-term expression of heterologous genes in vivo. Adenovirus is not associated with severe human pathology, the virus can infect a wide variety of cells and has a broad host-range, the virus can be produced in large quantities with relative ease, and the virus can be rendered replication defective by deletions in the early-region 1 (“E1”) of the viral genome. Thus, vectors derived from human adenoviruses, in which at least the E1 region has been deleted and replaced by a gene of interest, have been used extensively for gene therapy experiments in the pre-clinical and clinical phase.

Adenoviral vectors for use with the present invention are derived from any of the various adenoviral serotypes, including, without limitation, any of the over 40 serotype strains of adenovirus, such as serotypes 2, 5, 12, 40, and 41. The adenoviral vectors used herein are replication-deficient and contain the gene of interest under the control of a suitable promoter, such as any of the promoters discussed below with reference to adeno-associated virus. For example, U.S. Pat. No. 6,048,551, incorporated herein by reference in its entirety, describes replication-deficient adenoviral vectors that include the human gene for the anti-inflammatory cytokine IL-10, as well as vectors that include the gene for the anti-inflammatory cytokine IL-1ra, under the control of the Rous Sarcoma Virus (RSV) promoter, termed Ad.RSVIL-10 and Ad.RSVIL-1ra, respectively.

Other recombinant adenoviruses, derived from any of the adenoviral serotypes, and with different promoter systems, can be used by those skilled in the art. For example, U.S. Pat. No. 6,306,652, incorporated herein by reference in its entirety, describes adenovirus vectors with E2A sequences, containing the hr mutation and the ts125 mutation, termed ts400, to prevent cell death by E2A overexpression, as well as vectors with E2A sequences, containing only the hr mutation, under the control of an inducible promoter, and vectors with E2A sequences, containing the hr mutation and the ts125 mutation (ts400), under the control of an inducible promoter.

Moreover, “minimal” adenovirus vectors as described in U.S. Pat. No. 6,306,652 will find use with the present invention. Such vectors retain at least a portion of the viral genome that is required for encapsidation of the genome into virus particles (the encapsidation signal), as well as at least one copy of at least a functional part or a derivative of the ITR. Packaging of the minimal adenovirus vector can be achieved by co-infection with a helper virus or, alternatively, with a packaging-deficient replicating helper system as described in U.S. Pat. No. 6,306,652.

Other useful adenovirus-based vectors for delivery of anti-inflammatory cytokines include the “gutless” (helper-dependent) adenovirus in which the vast majority of the viral genome has been removed (Wu et al., Anesthes. (2001) 94:1119-1132). Such “gutless” adenoviral vectors essentially create no viral proteins, thus allowing virally driven gene therapy to successfully ensue for over a year after a single administration (Parks, R. J., Clin. Genet. (2000) 58:1-11; Tsai et al., Curr. Opin. Mol. Ther. (2000) 2:515-523) and eliminate interference by the immune system. In addition, removal of the viral genome creates space for insertion of control sequences that provide expression regulation by systemically administered drugs (Burcin et al., Proc. Natl. Acad. Sci. USA (1999) 96:355-360), adding both safety and control of virally driven protein expression. These and other recombinant adenoviruses will find use with the present methods.

Plasmid Gene Delivery Systems

As explained above, the gene of interest can be introduced into the subject or cells of the subject using non-viral vectors, such as plasmids, and any of the several plasmid delivery techniques well-known in the art. For example, vectors can be introduced without delivery agents, as described in, e.g., U.S. Pat. Nos. 6,413,942, 6,214,804 and 5,580,859, all incorporated by reference herein in their entireties.

Alternatively, the vectors encoding the gene of interest can be packaged in liposomes prior to delivery to the subject or to cells derived therefrom, such as described in U.S. Pat. Nos. 5,580,859; 5,549,127; 5,264,618; 5,703,055, all incorporated herein by reference in their entireties. Lipid encapsulation is generally accomplished using liposomes which are able to stably bind or entrap and retain nucleic acid. The ratio of condensed DNA to lipid preparation can vary but will generally be around 1:1 (mg DNA:micromoles lipid), or more of lipid. For a review of the use of liposomes as carriers for delivery of nucleic acids, see, Hug and Sleight, Biochim. Biophys. Acta. (1991) 1097:1-17; Straubinger et al., in Methods of Enzymology (1983), Vol. 101, pp. 512-527. The DNA can also be delivered in cochleate lipid compositions similar to those described by Papahadjopoulos et al., Biochem. Biophys. Acta. (1975) 394:483-491. See, also, U.S. Pat. Nos. 4,663,161 and 4,871,488, incorporated herein by reference in their entireties.

The vectors may also be encapsulated, adsorbed to, or associated with, particulate carriers, well known in the art. Such carriers present multiple copies of a selected molecule to the immune system and promote trapping and retention of molecules in local lymph nodes. The particles can be phagocytosed by macrophages and can enhance antigen presentation through cytokine release. Examples of particulate carriers include those derived from polymethyl methacrylate polymers, as well as microparticles derived from poly(lactides) and poly(lactide-co-glycolides), known as PLG. See, e.g., Jeffery et al., Pharm. Res. (1993) 10:362-368; and McGee et al., J. Microencap. (1996).

Moreover, plasmid DNA can be guided by a nuclear localization signal or like modification.

Additionally, biolistic delivery systems employing particulate carriers such as gold and tungsten, are useful for delivering genes of interest. The particles are coated with the gene to be delivered and accelerated to high velocity, generally under a reduced atmosphere, using a gun powder discharge from a “gene gun.” For a description of such techniques, and apparatuses useful therefore, see, e.g., U.S. Pat. Nos. 4,945,050; 5,036,006; 5,100,792; 5,179,022; 5,371,015; and 5,478,744, all incorporated herein by reference in their entireties.

A wide variety of other methods can be used to deliver the vectors. Such methods include DEAE dextran-mediated transfection, calcium phosphate precipitation, polylysine- or polyornithine-mediated transfection, or precipitation using other insoluble inorganic salts, such as strontium phosphate, aluminum silicates including bentonite and kaolin, chromic oxide, magnesium silicate, talc, and the like. Other useful methods of transfection include electroporation, sonoporation, protoplast fusion, peptoid delivery, or microinjection. See, e.g., Sambrook et al., supra, for a discussion of techniques for transforming cells of interest; and Felgner, P. L., Advanced Drug Delivery Reviews (1990) 5:163-187, for a review of delivery systems useful for gene transfer. Methods of delivering DNA using electroporation are described in, e.g., U.S. Pat. Nos. 6,132,419; 6,451,002, 6,418,341, 6233,483, U.S. Patent Publication No. 2002/0146831; and International Publication No. WO/0045823, all of which are incorporated herein by reference in their entireties.

It may also be desirable to fuse the plasmid encoding the gene of interest to immunoglobulin molecules in order to provide for sustained expression. One convenient technique is to fuse the plasmid encoding the agent of interest to the Fc portion of a mouse IgG2a with a noncytolytic mutation. Such a technique has been shown to provide for sustained expression of cytokines, such as IL-10, especially when combined with electroporation. See, e.g., Jiang et al., J. Biochem. (2003) 133:423-427; and Adachi et al., Gene Ther. (2002) 9:577-583.

Compositions and Delivery

A. Compositions

Once produced, the vectors (or virions) encoding the anti-inflammatory cytokine, will be formulated into compositions suitable for delivery. Compositions will comprise sufficient genetic material to produce a therapeutically effective amount of the anti-inflammatory cytokine of interest, i.e., an amount sufficient to reduce the symptoms of, or prevent the vascular disease in question. The compositions will also contain a pharmaceutically acceptable excipient. Such excipients include any pharmaceutical agent that does not itself induce the production of antibodies harmful to the individual receiving the composition, and which may be administered without undue toxicity. Pharmaceutically acceptable excipients include, but are not limited to, sorbitol, any of the various TWEEN compounds, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in REMINGTON'S PHARMACEUTICAL SCIENCES (Mack Pub. Co., N.J. 1991).

One particularly useful formulation comprises the vector or virion of interest in combination with one or more dihydric or polyhydric alcohols, and, optionally, a detergent, such as a sorbitan ester. See, for example, International Publication No. WO 00/32233.

As is apparent to those skilled in the art in view of the teachings of this specification, an effective amount can be empirically determined. Representative doses are detailed below. Administration can be effected in one dose, continuously or intermittently throughout the course of treatment. Methods of determining the most effective means and dosages of administration are well known to those of skill in the art and will vary with the vector, the composition of the therapy, the target cells, and the subject being treated. Single and multiple administrations can be carried out with the dose level and pattern being selected by the treating physician.

It should be understood that more than one transgene can be expressed by the delivered recombinant vector. For example, the recombinant vectors can encode more than one anti-inflammatory cytokine. Alternatively, separate vectors, each expressing one or more different transgenes, can also be delivered to glial cells as described herein. Thus, multiple anti-inflammatory cytokines can be delivered concurrently or sequentially.

Furthermore, it is also intended that the vectors delivered by the methods of the present invention be combined with other suitable compositions and therapies. For instance, other agents used to treat vascular disease, such as but not limited to beta blockers, calcium channel blockers, ACE inhibitors, angiotension II inhibitors such as angiotension II receptor antagonists, diuretics, tPA, reteplase, streptokinase, aspirin, vascular endothelial growth factor (VEGF), fibroblast growth factor (FGF), platelet-derived growth factor (PDGF), angiopoietin-1, and the like, can be coadministered with the compositions of the invention.

B. Delivery

The recombinant vectors may be introduced into cells and tissues of the subject using either in vivo or in vitro (also termed ex vivo) transduction techniques. If transduced in vitro, the desired recipient cell (for example, a muscle cell, such as a cell from skeletal muscle, smooth muscle e.g., cardiac muscle, myocytes such as myotubes, myoblasts, both dividing and differentiated, cardiomyocytes and cardiomyoblasts) will be removed from the subject, transduced with rAAV virions and reintroduced into the subject. Alternatively, syngeneic or xenogeneic cells can be used where those cells will not generate an inappropriate immune response in the subject. Suitable methods for the delivery and introduction of transduced cells into a subject have been described. For example, cells can be transduced in vitro by combining recombinant AAV virions with cells to be transduced in appropriate media, and those cells harboring the DNA of interest can be screened using conventional techniques such as Southern blots and/or PCR, or by using selectable markers. Transduced cells can then be formulated into pharmaceutical compositions, as described above, and the composition introduced into the subject by various techniques as described below, in one or more doses.

In one embodiment, rAAV virions or cells transduced in vitro are delivered directly to muscle by injection with a needle, catheter or related device, using techniques known in the art. In another embodiment, a catheter introduced into a peripheral artery (such as the femoral artery) can be used to deliver rAAV virions to a muscle of interest (such as cardiac muscle) via an artery that provides blood to the muscle of interest (such as the coronary artery which provides blood to the heart).

In another embodiment, rAAV virions or cells transduced in vitro are introduced into the bloodstream of the subject. Administration into the bloodstream may be by injection into a vein, an artery, or any other vascular conduit.

The rAAV virions or cells transduced in vitro may also be introduced into the subject by way of histamine or isolated limb perfusion. Isolated limb perfusion is a technique well known in the surgical arts, the method essentially enabling the artisan to isolate a limb from the systemic circulation prior to administration of the rAAV virions. See, e.g., Schaadt et al., J. Extra Corpor. Technol. (2002) 34:130-143; Lejeune et al., Surg. Oncol. Clin. N. Am. (2001) 10:821-832; Fraser et al., AORN J. (1999) 70:642-647, 649, 651-653. A variant of the isolated limb perfusion technique, described in U.S. Pat. No. 6,177,403 and incorporated herein by reference, can also be employed by the skilled artisan to administer the rAAV virions or cells transduced in vitro into the vasculature of an isolated limb to potentially enhance transduction into muscle cells or tissue.

Moreover, for certain conditions, it may be desirable to deliver the rAAV virions or cells transduced in vitro to the CNS of a subject. By “CNS” is meant all cells and tissue of the brain and spinal cord of a vertebrate. Thus, the term includes, but is not limited to, neuronal cells, glial cells, astrocytes, cereobrospinal fluid (CSF), interstitial spaces, bone, cartilage and the like. Recombinant AAV virions or cells transduced in vitro may be delivered directly to the CNS or brain by injection into, e.g., the ventricular region, as well as to the striatum (e.g., the caudate nucleus or putamen of the striatum), spinal cord and neuromuscular junction, or cerebellar lobule, with a needle, catheter or related device, using neurosurgical techniques known in the art, such as by stereotactic injection (see, e.g., Stein et al., J Virol 73:3424-3429, 1999; Davidson et al., PNAS 97:3428-3432, 2000; Davidson et al., Nat. Genet. 3:219-223, 1993; and Alisky and Davidson, Hum. Gene Ther. 11:2315-2329, 2000).

One mode of administration of recombinant AAV virions uses a convection-enhanced delivery (CED) system. In this way, recombinant virions can be delivered to many cells over large areas of muscle or tissue. Any convection-enhanced delivery device may be appropriate for delivery of viral vectors. In a preferred embodiment, the device is an osmotic pump or an infusion pump. Both osmotic and infusion pumps are commercially available from a variety of suppliers, for example Alzet Corporation, Hamilton Corporation, Alza, Inc., Palo Alto, Calif.). Typically, a viral vector is delivered via CED devices as follows. A catheter, cannula or other injection device is inserted into appropriate muscle tissue in the chosen subject, such as skeletal muscle. For a detailed description regarding CED delivery, see U.S. Pat. No. 6,309,634, incorporated herein by reference in its entirety.

The dose of rAAV virions required to achieve a particular therapeutic effect, e.g., the units of dose in vector genomes (vg), will vary based on several factors including, but not limited to: the species, the route of rAAV virion administration, the level of heterologous nucleic acid sequence expression required to achieve a therapeutic effect, the specific disease or disorder being treated, a host immune response to the rAAV virion, a host immune response to the heterologous nucleic acid sequence expression product, and the stability of the expression product. One skilled in the art can readily determine a rAAV virion dose range to treat a patient having a particular disease or disorder based on the aforementioned factors, as well as other factors.

A therapeutically effective dose will include on the order of from about 10⁸ to 10²⁰ of the rAAV virions, more preferably 10¹⁰ to 10¹⁴, and even more preferably about 10¹¹ to 10¹³ of the rAAV virions (or viral genomes, also termed “vg”), or any value within these ranges.

Generally, from 1 μl to 1 ml of composition will be delivered, such as from 0.01 to about 0.5 ml, for example about 0.05 to about 0.3 ml, such as 0.08, 0.09, 0.1, 0.2, etc. and any number within these ranges, of composition will be delivered.

### 3. EXPERIMENTAL

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### EXAMPLE 1

### Effect of rAAV-IL-10 on C2C12 Mouse Myoblasts

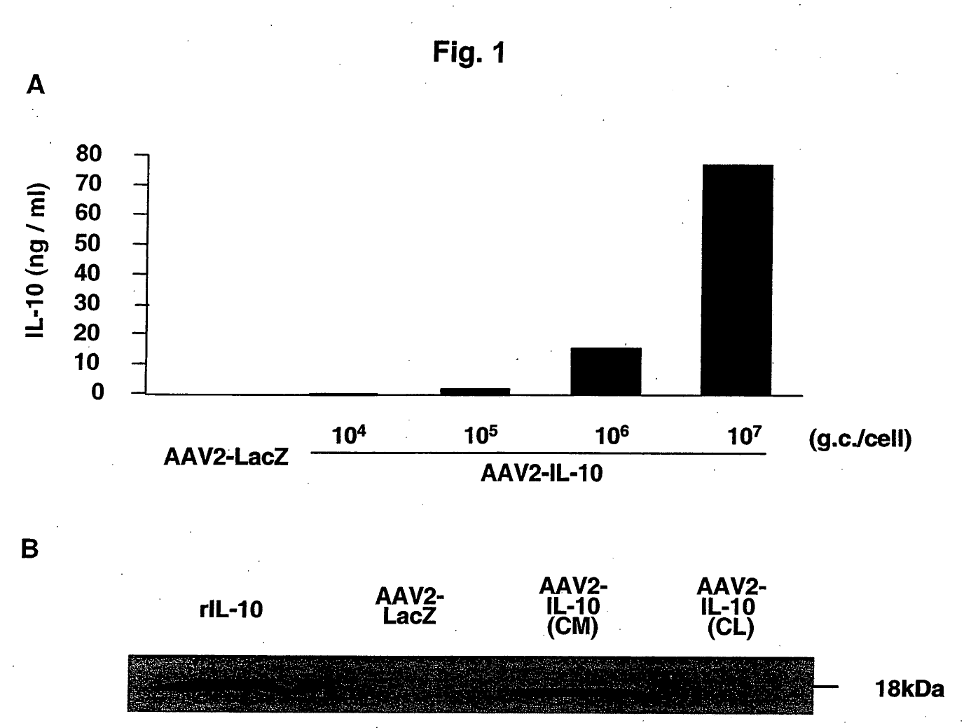

In order to determine the ability of gene-delivered IL-10 to treat or prevent atherosclerosis, the following in vitro experiment was conducted. C2C12 mouse myoblasts were cultured in a well of 6-well plate with 2 ml of DMEM containing 5% horse serum. Eight days after plating, differentiated C2C12 cells were infected with recombinant AAV2 virions encoding mouse IL-10 (rAAV2-mIL-10) or rAAV2-LacZ as a control, at various multiplicities of infection (MOI) ranging from approximately 1×10⁴ to 1×10⁷ genome copies/cell. The expression of mIL-10 was detected by Western blot analysis after immunoprecipitation of the conditioned medium and cell lysate (FIG. 1B).

IL-10 levels were evaluated by ELISA 48 hours after infection. As shown in FIG. 1B, IL-10 production increased in a dose-dependent manner in the IL-10 transduced C2C12 conditioned medium.

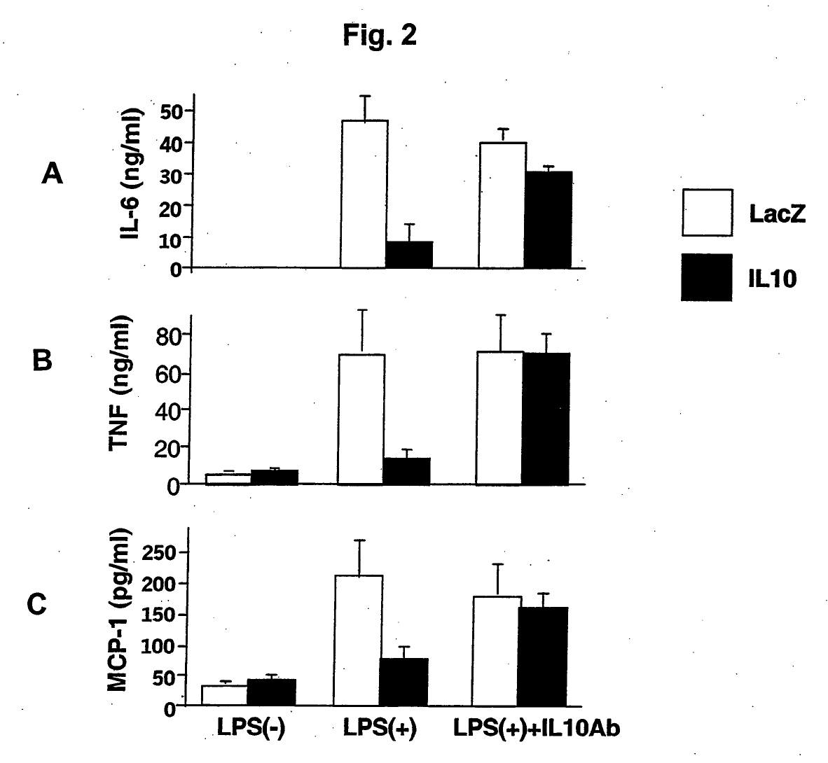

The conditioned medium was diluted with DMEM to 10 ng/ml and was put on J774 mouse macrophages for 30 min. After the pretreatment, J774 mouse macrophages were treated with 100 ng/ml lipopolysaccharides (LPS) to induce proinflammatory cytokine production and incubated for an additional 24 hr in the presence or absence of anti-IL-10 antibody. Supernatants were harvested and production of the proinflammatory cytokines IL-6, TNF-α, and MCP-1 by J774 mouse macrophages was quantified by ELISA to evaluate the effect of secreted IL-10. As shown in FIGS. 2A-2C, LPS-induced production of the proinflammatory cytokines by J774 cells was significantly suppressed in the rAAV-IL-10 group and was abolished in the presence of anti-mIL-10 antibody.

Thus, myocytes transduced with rAAV-IL-10 efficiently secreted IL-10. Moreover, rAAV-delivered IL-10 effectively inhibited the inflammatory response of macrophages in vitro.

### EXAMPLE 2

### Ability of rAAV-IL-10 to Modulate the Atherosclerotic Process

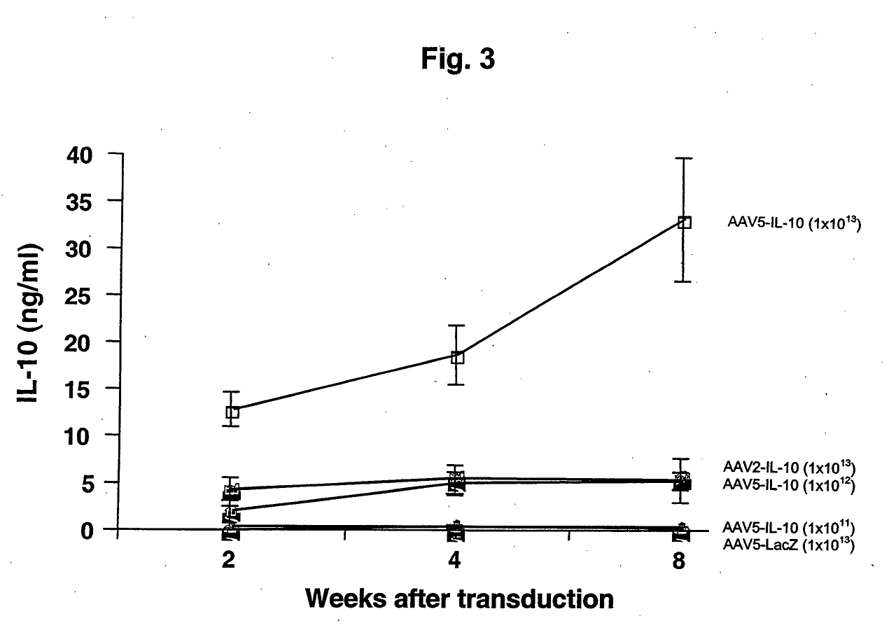

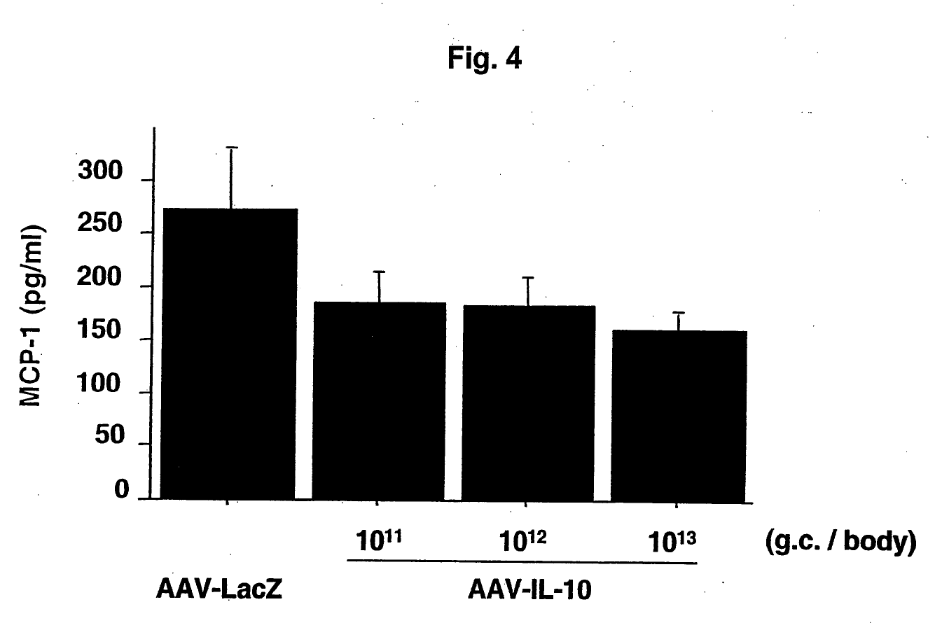

In order to determine the ability of gene-delivered IL-10 to treat or prevent atherosclerosis, the following in vivo experiment was conducted. ApoE-deficient mice were obtained from Banyu Pharmaceutical Co., Ltd. (by the courtesy of Dr. Ishibashi), and were fed a western diet containing 21% fat and 0.15% cholesterol (Harlan TEKLAD) from 1 month of age. Mice were kept in accordance with standard animal care requirements, and maintained a 12-hour light-dark cycle. Water and food were given ad libitum. Apo E-deficient mice at 2 months of age were infected with rAAV2-mIL-10 (1×10¹³ particles/body), rAAV5-mIL-10 (1×10¹¹ to 10¹³ particles/body), or rAAV5-LacZ (1×10¹³ particles/body) as a control into the anterior tibial muscles. 2, 4, and 8 weeks after the inoculation, serum IL-10 concentration was monitored. As shown in FIG. 3, IL-10 gene transfer resulted in a significant dose-dependent increase of serum IL-10 levels which was maintained for at least 8 weeks. Serum MCP-1 levels were also measured. As shown in FIG. 4, serum MCP-1 levels were reduced in mice transduced with rAAV-IL-10.

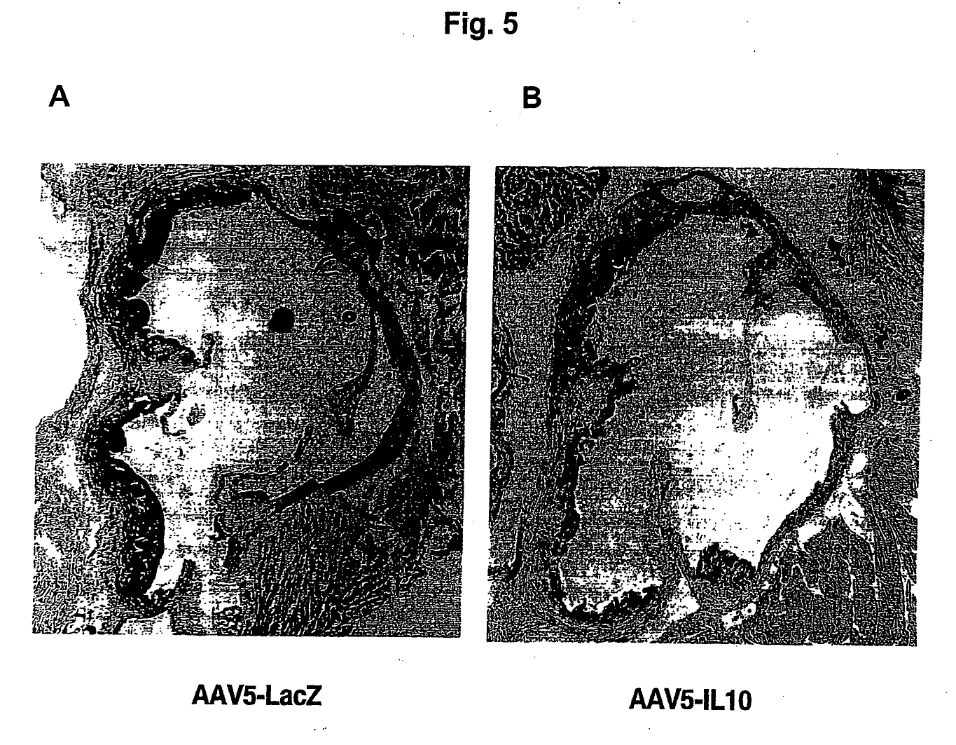

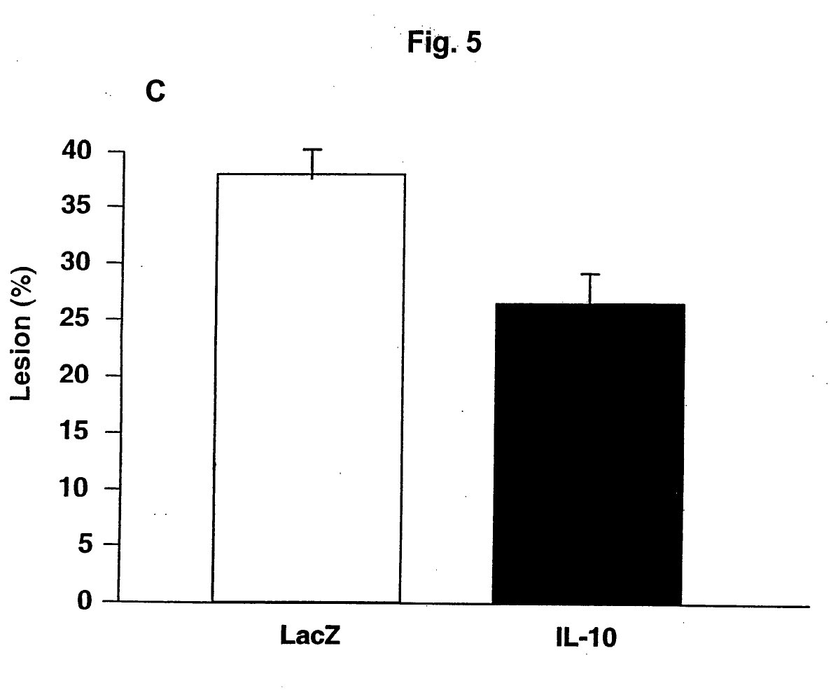

Eight weeks after rAAV infection, the ascending aortas were removed after perfusion fixation with 4% paraformaldehyde at physiological pressure, embedded in OCT compound (Tissue-Tek, Tokyo, Japan), and frozen in liquid nitrogen. Atherosclerotic lesions in the aortic sinus region were examined at five locations, each separated by 80 μm, with the most proximal site starting where the aortic valves first appeared, and were stained with oil red-O. To quantify the atherosclerotic lesions, each image was digitized and analyzed with an Olympus microscope and National Institutes of Health Image software. See, FIG. 5A. Lipid lesion formation was analyzed by determining the percent area of oil red-O stained to total cross-sectional vessel wall area. See, FIG. 5B. The average value for the five locations for each animal was used for analysis. As shown in FIG. 5C, rAAV-IL-10 transduction resulted in 31% reduction of the atherosclerotic area (R=0.65).

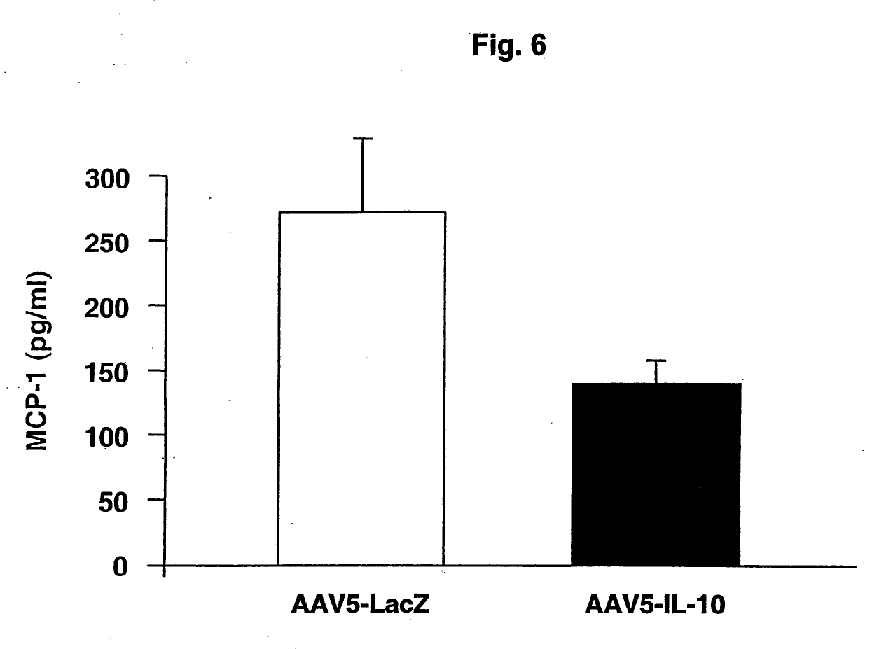

Serum MCP levels were measured 8 weeks after infection of the apoE deficient mice with rAAV5-IL-10 or rAAV5-LacZ as a control, using an ELISA. As shown in FIG. 6, MCP-1 levels were reduced in mice administered rAAV5-IL-10 relative to control mice.

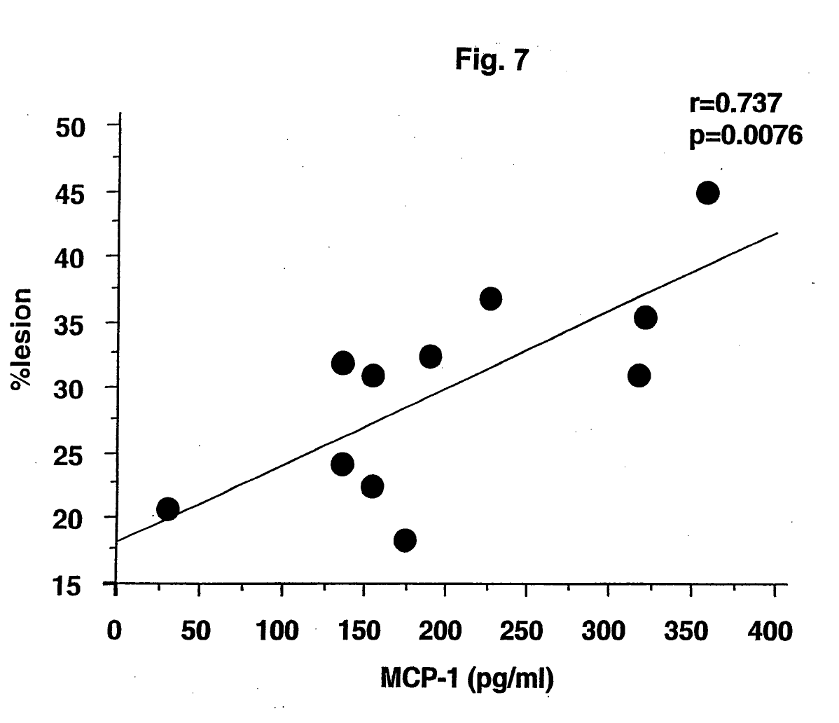

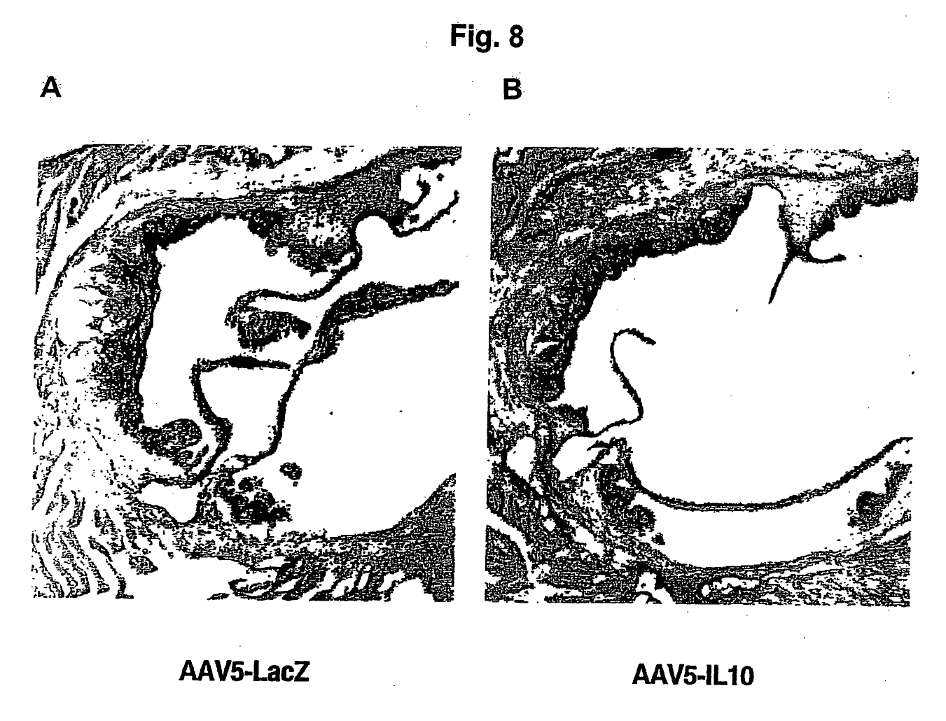

To examine infiltration of inflammatory cell and secretion of cytokines, immunohistochemical staining was performed. Arterial sections were obtained 8 weeks after injection of rAAV5-IL-10 or rAAV-LacZ and prepared as described above, blocked endogenous peroxidase and non-reacting binding site on the secondary antibody, and then incubated with a primary goat polyclonal antibody against murine MCP-1 (dilution 1/250; Santa Cruz Biotechnology, California, USA). Non-immune IgGs were used for negative controls. After incubation with biotinylated anti-mouse secondary antibody followed by peroxidase-conjugated streptavidin, 3′,3′-diaminobenzidine (DAB) was used as enzyme substrates. Results are shown in FIGS. 8A and 8B. As seen in FIG. 7, there was a significant positive correlation between MCP-1 level and the lesion size (r=0.737, p<0.01).

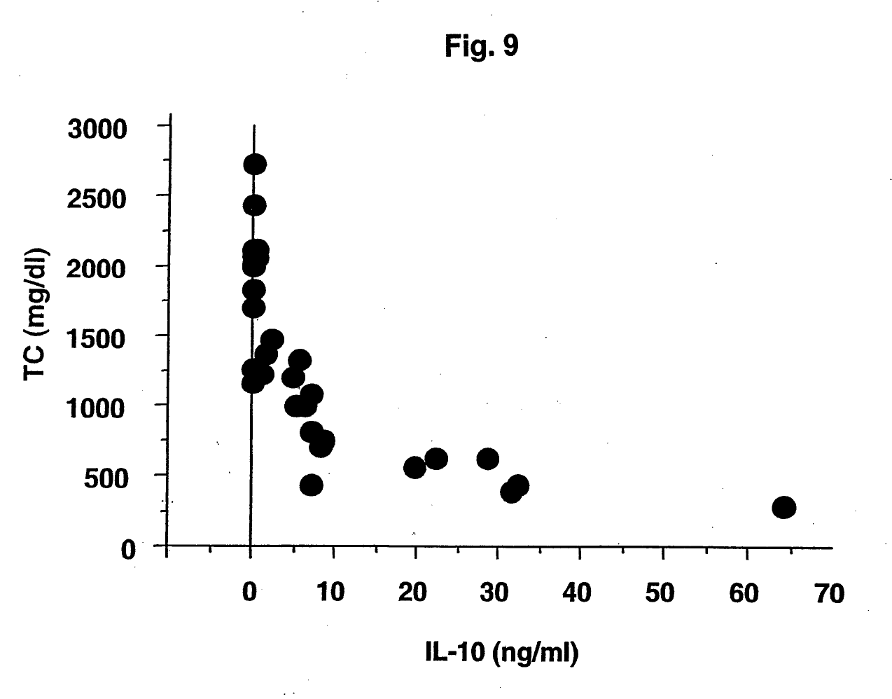

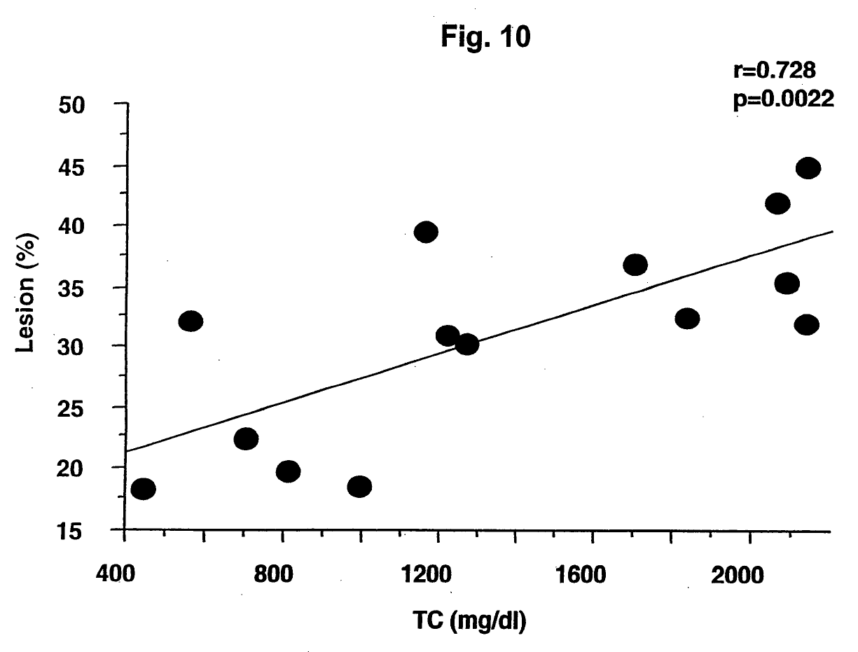

Serum cholesterol levels were measured and compared to serum IL-10 levels and atherosclerotic lesions. As shown in FIG. 9, there was a positive correlation between serum cholesterol levels and serum IL-10 concentration. As shown in FIG. 10, the serum cholesterol level correlated with atherosclerotic lesion (r=0.728, p<0.01).

Therefore, intramuscular injection of rAAV-IL-10 provided for sustained IL-10 expression along with inhibition of the atherosclerotic process.

### EXAMPLE 3

### Ability of rAAV-IL 10 to Reduce Blood Pressure and Stroke Episode

To test for the ability of anti-inflammatory molecules such as IL-10 to reduce hypertensive arterial damage and reduce blood pressure and strokes, the effect of gene-delivered IL-10 on stroke-prone spontaneously hypertensive rats (SHR-SP) was examined. This animal model is widely used to study hypertensive cerebrovascular disorders and displays severe hypertension, stroke episodes and renal interstitial inflammation.

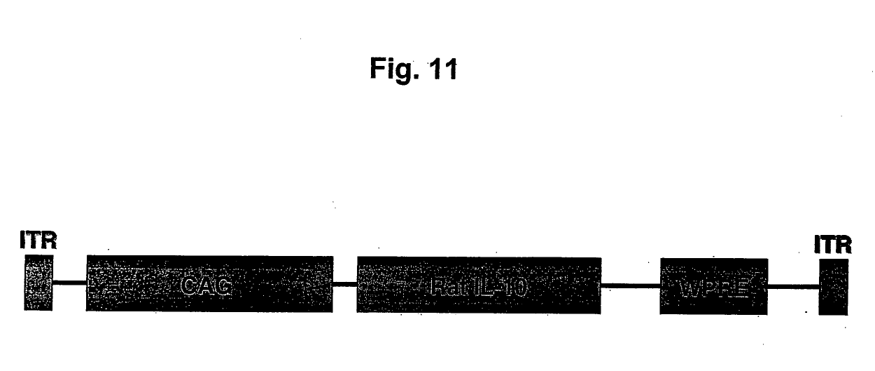

In particular, rat IL-10 was cloned from rat splenocytes cDNA by RT-PCR using the following primers, 5′-GCACGAGAGCCACAACGCA (SEQ ID NO: 1), 5′-GATTTGAGTACGATCCATTTATTCAAAACGAGGAT (SEQ ID NO:2). The 1.3 kb PCR fragment was cloned into pCR2.1 (pCR2.1 TOPO; Invitrogen, Inc.) by the TA cloning method. The PCR-amplified fragment was verified by sequencing both strands. Resultant plasmid pCR2.1RatIL-10 was digested with EcoRI, and the IL-10 gene fragment was inserted into the EcoRI site of p3.3CAG-WPRE which contains the CAG promoter and the woodchuck posttranscriptional regulatory element (WPRE). Next, the entire expression cassette was inserted between the ITRs of a pUC-based proviral plasmid to produce plasmid pWCAGRIL10W. See, FIG. 11.

Recombinant AAV viral stocks were propagated according to a three-plasmid transfection protocol. Briefly, 60% confluent 293 cells were cotransfected with the proviral plasmid, AAV helper plasmid p1RepCap (for rAAV1) or p5RepCap (for rAAV5), and adenoviral helper plasmid. Resultant viruses (rAAV1IL-10, rAAV5IL-10 or control vectors expressing EGFP) were purified through two rounds of CsCl two-tier centrifugation. The physical titer of the viral stock was determined by dot blot hybridization with plasmid standards.

293 cells were transfected with pWCAGRIL10 or pW1 (containing LacZ) using calcium phosphate. The supernatant and the cell lysate were collected 48 hours after transfection. These samples were subjected to electropheresis on 10% SDS-PAGE under reducing conditions and transferred to a nitrocellulose membrane. The membrane was blocked and incubated with mouse anti-rat IL-10. The membrane was rinsed and incubated with peroxidase-linked anti-mouse IgG antibody. Immunoreactive bands were visualized using the ECL Western blotting kit. A 19 kDa protein was seen in the pWCAGRIL10-transfected supernatant of 293 cells. This result is consistent with the molecular weight and secretory property of rat IL-10.

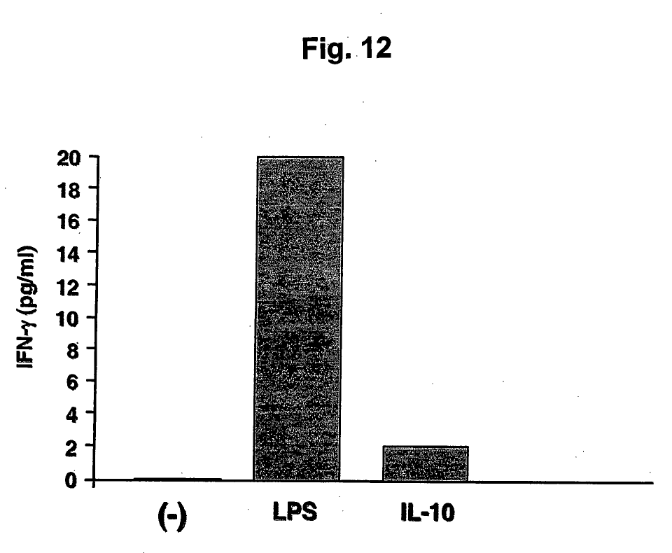

The biological activity of rat IL-10 was determined as follows. 293 cells were transduced with rAAV virions encoding rat IL-10. Forty-eight hours after infection, supernatant was recovered and the concentration of rat IL-10 was determined by ELISA. After the concentration of IL-10 was adjusted to 2 ng/ml, the supernatant was incubated with rat primary monocytes. 30 minutes after incubation, LPS was added at the concentration of 10 ng/ml. 24 hours later, the concentration of interferon-γ in the supernatant was determined by ELISA. As can be seen in FIG. 12, the supernatant from AAV-IL-10 infected 293 cells inhibited the production of interferon-γ, indicating that the rat IL-10 was biologically active.

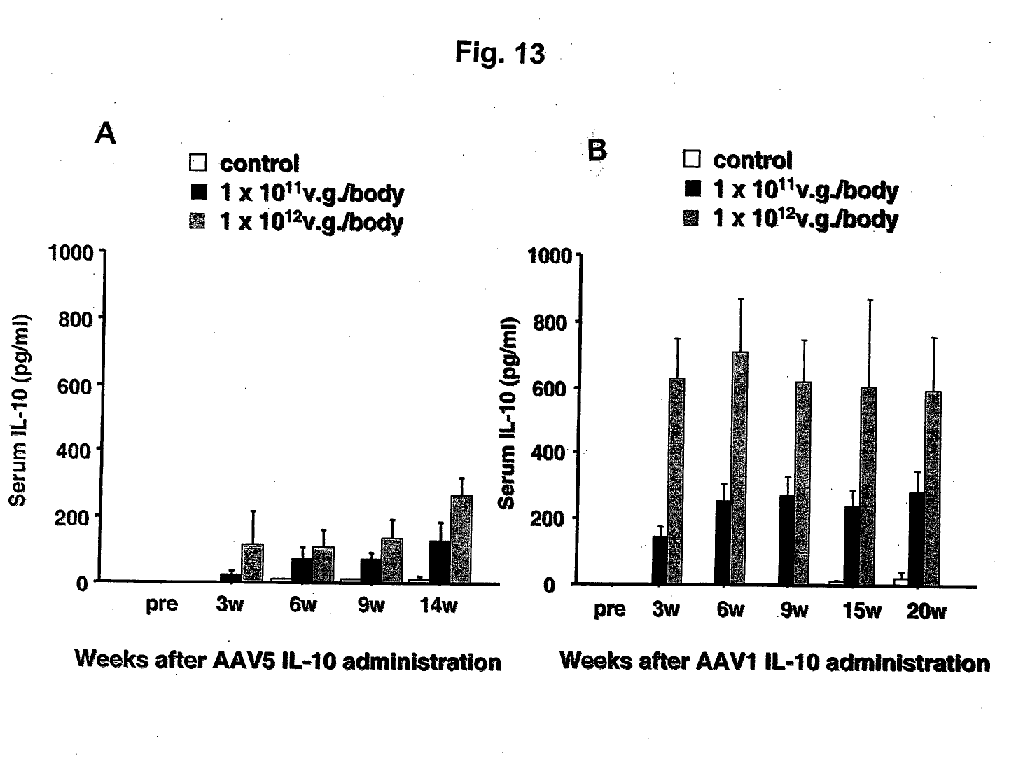

Male SHR-SP rats were administered rAAV1-IL-10 (1×10¹¹ v.g. or 1×10¹² v.g./body), rAAV5-IL-10 (1×10¹¹ v.g. or 1×10¹² v.g./body), control vector, or saline (n=5 for each group) in the bilateral anterior tibial muscles at 6 weeks of age. At 8 weeks of age, rats were fed special chow. The serum concentration of IL-10 was determined by ELISA periodically. As seen in FIGS. 13A and 13B, the serum concentration of IL-10 increased in a vector dose-dependent manner and the transduction efficiency was higher with AAV1 than AAV5.

Systolic blood pressure was measured every week in male SHR-SP rats administered rAAV1-IL-10, rAAV5-IL-10, or a control (n=10 for each group) at six weeks of age in the bilateral anterior tibial muscles. Blood pressure was determined by the tail-cuff method. Twenty hour urine samples were collected using metabolic cages and proteinuria was evaluated 9 weeks after viral injection. Echocardiogram was performed 14 weeks after transduction. Ejection fraction (EF) was evaluated 14 weeks after transduction. Left ventricle end-diastolic dimension (LVEDD) and left ventricle end-systolic dimension (LVESD) were measured in parasternal long-axis view at the level between the papillary muscle and mitral leaflet tips. Left ventricle volume (V) was determined by the dimension (D) using Teichholz's equation. V=[7.0/(2.4+D)]×D3. Stroke volume (SV) equals LVEDV−LVESV; EF equals SV/LVEDV. The incidence of stroke-associated symptoms was also assessed as a physiological parameter. Seizure, paralysis of hind limb, and decreased activity were considered symptoms of stroke. Rats were monitored for behavioral assessment every day. The percentage of stroke-free animals was evaluated by the Kaplan-Meier method. Nine weeks after transduction, the serum concentration of IL-10 and TGF-β were determined by ELISA.

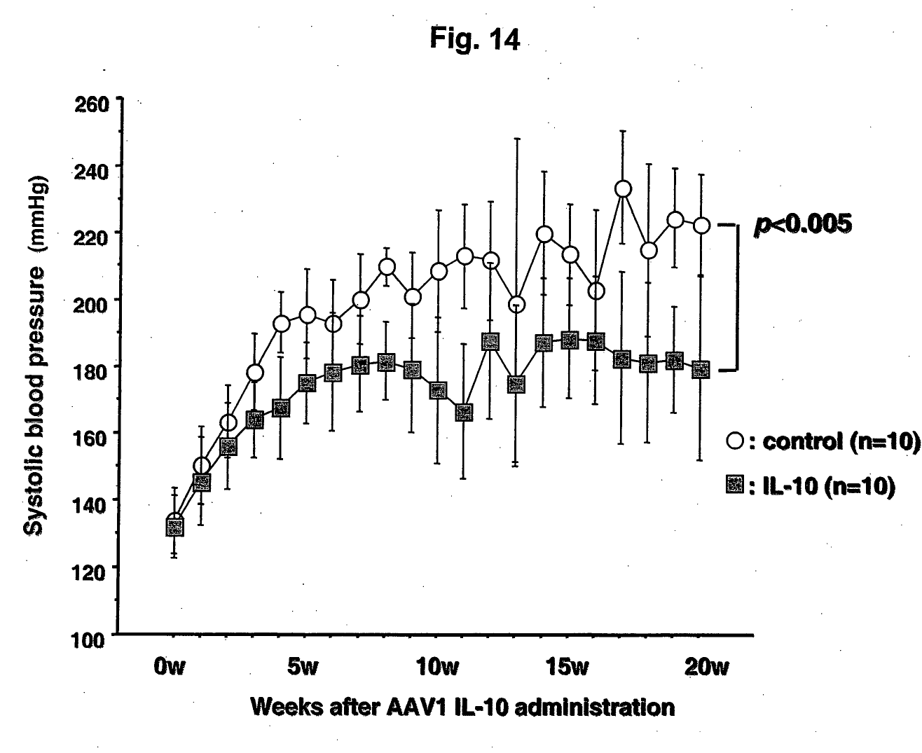

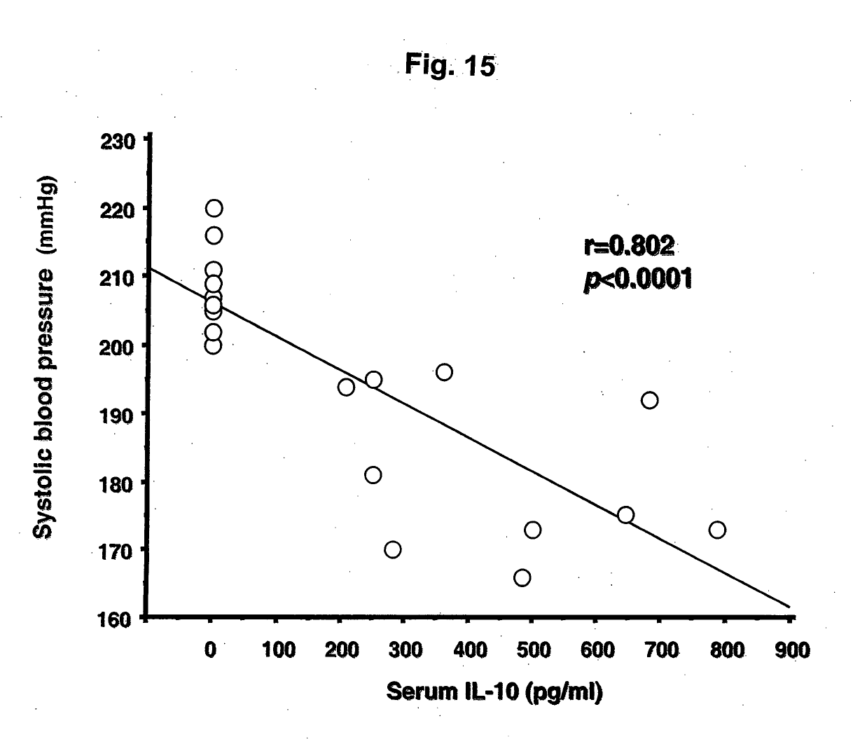

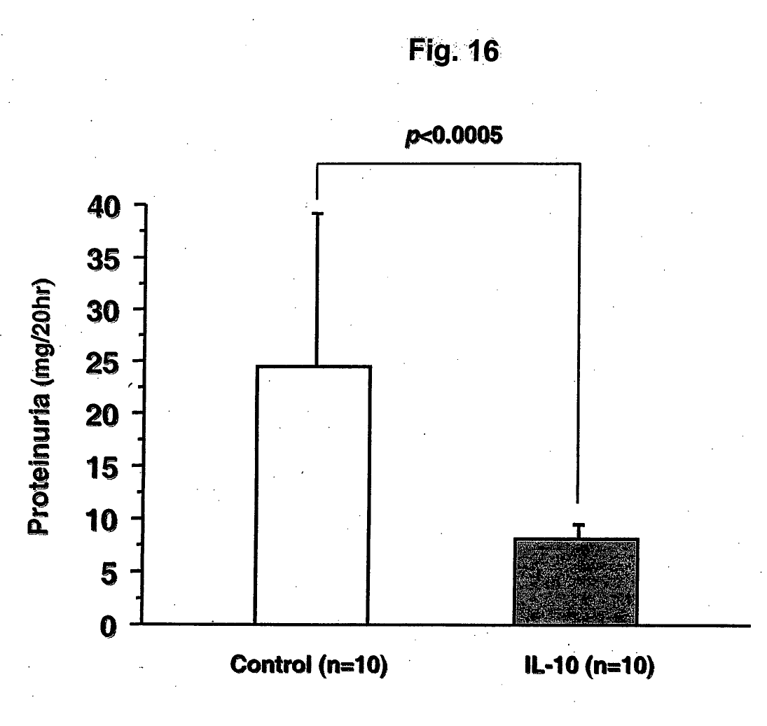

As seen in FIG. 14, three weeks following rAAV1-IL -10 administration, blood pressure significantly decreased relative to the control group and the reduction persisted for at least 20 weeks (175±9.6 mmHg vs. 205±2.5 mmHg at 8 weeks, p<0.01). As shown in FIG. 15, the serum concentration of IL-10 also significantly correlated with the decrease in blood pressure (r+0.59, p<0.005). Similarly, three weeks after administration of rAAV5-IL-10, blood pressure decreased in comparison to the control group (162±2.0 mmHg vs. 181±10.8 mmHg, p<0.05). Proteinuria was also decreased at 9 weeks after transduction relative to controls (see, FIG. 16), indicating a decrease in renal damage.

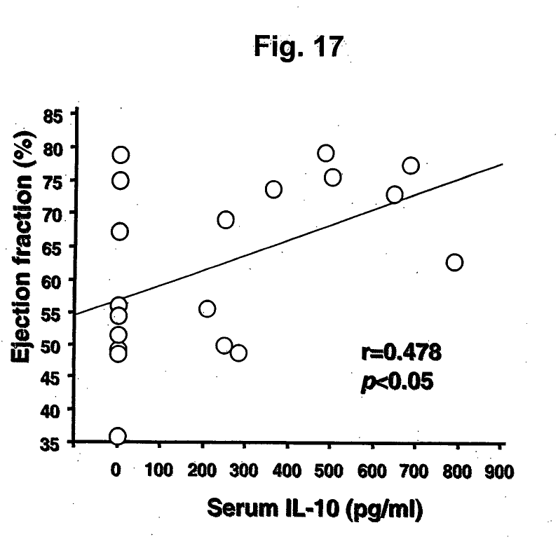

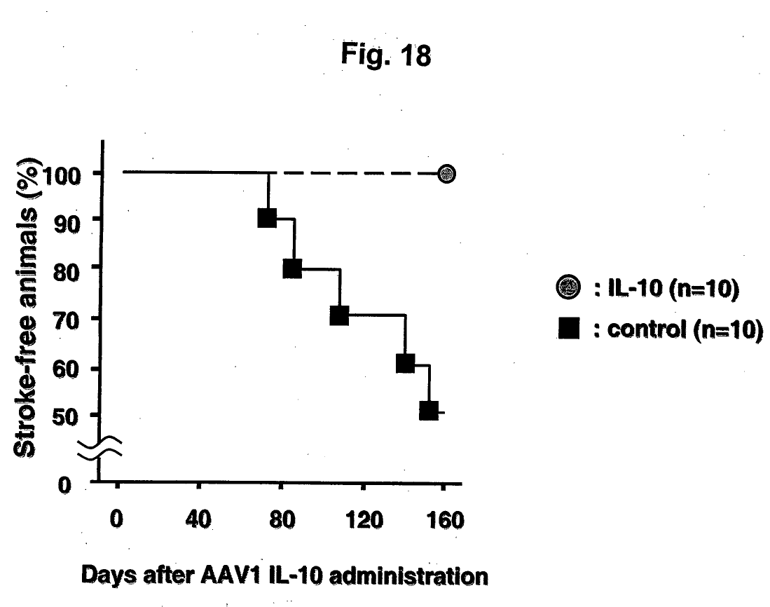

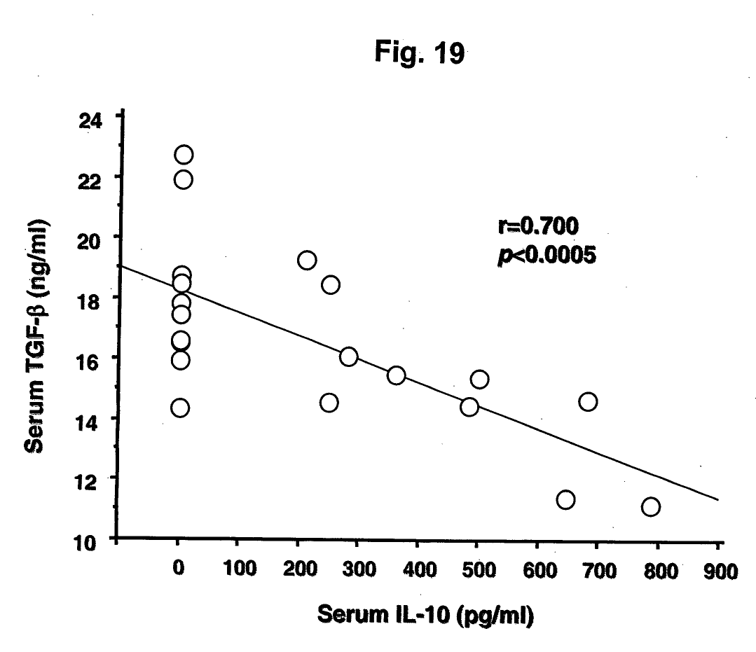

FIG. 17 shows the correlation between ejection fraction and serum IL-10 concentration (r=0.478, p<0.05). As shown in FIG. 18, stroke episode was decreased in SHR-SP animals administered rAAV1IL-10 as compared to the control group. Stroke episode was also significantly decreased in the animals administered rAAV5IL-10 (p<0.05). Moreover, as serum IL-10 levels increased, there was a down-regulation of serum TGF-β (r=0.700, p<0.0005).

In summary, AAV-mediated IL-10 gene transfer reduced blood pressure over 20 weeks. There was a tight correlation between IL-10 concentration and blood pressure. IL-10 gene transfer also decreased proteinuria and prolonged stroke-free duration. Without being bound by a particular theory, renal protection through down-regulation of TGF-β may be involved in these beneficial effects. Taken together, the above data show that rAAV-mediated IL-10 gene transfer is effective for treating and preventing hypertension as well as hypertension-related organ damage.

Thus, methods for delivering anti-inflammatory cytokines for the treatment and prevention of vascular disease and vascular disease-related organ damage are described. Although preferred embodiments of the subject invention have been described in some detail, it is understood that obvious variations can be made without departing from the spirit and the scope of the invention as defined herein.

We claim:

1. A method of treating or preventing vascular disease in a vertebrate subject comprising administering to said subject a composition comprising a recombinant virus, wherein the recombinant virus comprises a polynucleotide encoding interleukin-10 (IL-10), operably linked to expression control elements, under conditions that result in expression of said polynucleotide in vivo to provide a therapeutic effect.

2. The method of claim 1, wherein said vertebrate subject is a human and said IL-10 is human IL-10.

3. The method of claim 1, wherein said recombinant virus is a recombinant adeno-associated virus virion.

4. The method of claim 2, wherein said recombinant virus is a recombinant adeno-associated virus virion.

5. The method of claim 1, wherein said administering is by direct delivery to a vascular conduit of said subject.

6. A method of treating or preventing vascular disease in a mammalian subject, comprising intramuscularly administering to said subject a composition comprising a recombinant virus, wherein the recombinant virus comprises a polynucleotide encoding interleukin-10 (IL-10), operably linked to expression control elements, under conditions that result in expression of said polynucleotide in vivo to produce a therapeutic effect.

7. The method of claim 6, wherein the vascular disease is arteriolosclerosis.

8. The method of claim 6, wherein the vascular disease is atherosclerosis.

9. The method of claim 6, wherein the vascular disease is stroke.

10. The method of claim 6, wherein the vascular disease is hypertension.

11. The method of claim 6, wherein said mammalian subject is a human and said IL-10 is human IL-10.

12. The method of claim 11, wherein said recombinant virus is a recombinant adeno-associated virion.

13. A method of treating or preventing atherosclerosis in a mammalian subject, comprising intramuscularly administering to said subject a composition comprising a recombinant adeno-associated virus virion, wherein said virion comprises a polynucleotide encoding IL-10, operably linked to expression control elements, under conditions that result in expression of said polynucleotide in vivo to produce a therapeutic effect.

14. A method of reducing the incidence of stroke in a mammalian subject, comprising intramuscularly administering to said subject a composition comprising a recombinant adeno-associated virus virion, wherein said virion comprises a polynucleotide encoding IL-10, operably linked to expression control elements, under conditions that result in expression of said polynucleotide in vivo to produce a therapeutic effect.

15. A method of treating or preventing hypertension in a mammalian subject, comprising intramuscularly administering to said subject a composition comprising a recombinant adeno-associated virus virion, wherein said virion comprises a polynucleotide encoding IL-10, operably linked to expression control elements, under conditions that result in expression of said polynucleotide in vivo to produce a therapeutic effect.


SEQUENCE LISTING (2 sequences)

SEQ ID NO: 1 - DNA, 19 bp, Artificial
  rat IL-10 RT-PCT primer
gcacgagagccacaacgca

SEQ ID NO: 2 - DNA, 35 bp, Artificial
  rat IL-10 RT-PCT primer
gatttgagtacgatccatttattcaaaacgaggat